(19) United States
(12) Patent Application Publication
(10) Pub. No.: US 2026/0285787 A1
(43) Pub. Date: Sep. 24, 2026

(54) AZEOTROPE AND AZEOTROPE-LIKE COMPOSITIONS OF 1,1-DIFLUOROPROPENE (HFO-1252ZC) AND OTHER COMPOUNDS

(71) Applicant: Solstice Advanced Materials US, Inc., Morris Plains, NJ (US)

(72) Inventors: Joshua Close, Cheektowaga, NY (US); Pramod K.W. Harikumar Warrier, Cheektowaga, NY (US); Ryan J. Hulse, Getzville, NY (US); Haiyou Wang, Amherst, NY (US); Dimitrios Papanastasiou, Buffalo, NY (US); Justin Howard, Lakeview, NY (US)

(21) Appl. No.: 19/571,253

(22) Filed: Mar. 18, 2026

Related U.S. Application Data
(60) Provisional application No. 63/774,303, filed on Mar. 19, 2025.

(51) Int. Cl. C07C 17/383; C07C 21/18

(52) U.S. Cl. CPC C07C 17/383; C07C 21/18

(57) ABSTRACT

An azeotrope or azeotrope-like compositions consisting essentially of effective amounts of 1,1-difluoropropene (HFO-1252zc) and another compound including each of chlorotrifluoroethylene (CTFE), hexafluoropropylene (HFP), and cis-1,2-difluoroethylene (HFO-1132(Z)), and methods for separating the azeotrope or azeotrope-like compositions and/or exploiting the compositions in connection with methods of manufacturing 1,1-difluoropropene (HFO-1252zc).

### CROSS-REFERENCE TO RELATED APPLICATIONS

This application claims priority to U.S. Provisional Application No. 63/774,303, filed Mar. 19, 2025, which is herein incorporated by reference its entirety.

### FIELD

The present disclosure relates to azeotrope and azeotrope-like compositions and, in particular, to azeotrope and azeotrope-like compositions consisting essentially of 1,1-difluoropropene (HFO-1252zc) and another compound, including each of chlorotrifluoroethylene (CTFE), hexafluoropropylene (HFP), and/or cis-1,2-difluoroethylene (HFO-1132(Z)).

### BACKGROUND

Fluorocarbon fluids have properties that are desirable for use as heat transfer media, immersion coolants, liquid or gaseous dielectrics, industrial refrigerants, and other applications.

For example, 1,1-difluoropropene (HFO-1252zc) has recently found increased utility for a variety of uses. Potential end use applications of 1,1-difluoropropene (HFO-1252zc) include refrigerants, either used alone or in blends with other components, solvents for organic materials, and as a chemical intermediate in the synthesis of other halogenated hydrocarbon solvents. Improved methods for the production of 1,1-difluoropropene (HFO-1252zc) are desired.

Azeotrope and azeotrope-like compositions may be encountered during the manufacture of fluorocarbon fluids and understanding any such azeotrope or azeotrope-like compositions is helpful to improve the efficiency of the manufacturing processes.

### SUMMARY

The present disclosure provides minimum-boiling, homogenous azeotrope or azeotrope-like compositions consisting essentially of effective amounts of 1,1-difluoropropene (HFO-1252zc) and another compound including each of chlorotrifluoroethylene (CTFE), hexafluoropropylene (HFP), cis-1,2,3,3,3-pentafluoro propene (HFO-1225ye(Z)), HF, water (H₂O), chloroethyne (C2HCl), trans-1,2-difluoropropene (HFO-1252ye(E)), 1-chloro-1-fluoroethylene (HCFO-1131a), 3,3,3-trifluoropropene (HFO-1243zf), and/or cis-1,2-difluoroethylene (HFO-1132(Z)), and methods for separating the azeotrope or azeotrope-like compositions and/or exploiting the compositions in connection with methods of manufacturing 1,1-difluoropropene (HFO-1252zc).

In one form thereof, the present disclosure provides an azeotrope or azeotrope-like composition consisting essentially of effective amounts of 1,1-difluoropropene (HFO-1252zc) and chlorotrifluoroethylene (CTFE).

In another form thereof, the present disclosure provides an azeotrope or azeotrope-like composition consisting essentially of effective amounts of 1,1-difluoropropene (HFO-1252zc) and hexafluoropropylene (HFP).

In another form thereof, the present disclosure provides an azeotrope or azeotrope-like composition consisting essentially of effective amounts of 1,1-difluoropropene (HFO-1252zc) and cis-1,2-difluoroethylene (HFO-1132(Z)).

In another form thereof, the present disclose provides a method of separating and azeotrope or azeotrope like composition comprising 1,1-difluoropropene (HFO-1252zc) and another compound, such as chlorotrifluoroethylene (CTFE), hexafluoropropylene (HFP), and/or cis-1,2-difluoroethylene (HFO-1132(Z)).

### BRIEF DESCRIPTION OF THE DRAWINGS

FIG. 1 corresponds to Example 1 and is a graph of PTx measurements for an azeotrope or azeotrope-like composition of 1,1-difluoropropene (HFO-1252zc) and chlorotrifluoroethylene (CTFE) at an average temperature of −9.3° C.

FIG. 2 corresponds to Example 2 and is a graph of PTx measurements for an azeotrope or azeotrope-like composition of 1,1-difluoropropene (HFO-1252zc) and cis-1,2-difluoroethylene (HFO-1132(Z)) at an average temperature of 0.2° C.

FIG. 3 corresponds to Example 3 and is is a graph of PTx measurements for an azeotrope or azeotrope-like composition of hexafluoropropylene (HFP) and 1,1-difluoropropene (HFO-1252zc) at an average temperature of 0.4° C. FIG. 12 is a schematic of an apparatus for the separation by pressure swing distillation of an azeotrope or azeotrope-like composition of 1,1-difluoropropene (HFO-1252zc) and another compound, including each of chlorotrifluoroethylene (CTFE), hexafluoropropylene (HFP), and/or cis-1,2-difluoroethylene (HFO-1132(Z)).

FIG. 4 is a schematic of an apparatus for the separation by pressure swing distillation of an azeotrope or azeotrope-like composition of 1,1-difluoropropene (HFO-1252zc) and another compound, including each of chlorotrifluoroethylene (CTFE), hexafluoropropylene (HFP), and/or cis-1,2-difluoroethylene (HFO-1132(Z)).

FIG. 5 is a schematic of an apparatus for the separation by extractive distillation of an azeotrope or azeotrope-like composition of 1,1-difluoropropene (HFO-1252zc) and another compound, including each of chlorotrifluoroethylene (CTFE), hexafluoropropylene (HFP), and/or cis-1,2-difluoroethylene (HFO-1132(Z)).

### DETAILED DESCRIPTION

The present disclosure provides azeotropic or azeotrope-like compositions consisting essentially of 1,1-difluoropropene (HFO-1252zc) and at least one of chlorotrifluoroethylene (CTFE), hexafluoropropylene (HFP), and/or cis-1,2-difluoroethylene (HFO-1132(Z)).

The present disclose also provides schemes for separating 1,1-difluoropropene (HFO-1252zc) and at least one of chlorotrifluoroethylene (CTFE), hexafluoropropylene (HFP), and/or cis-1,2-difluoroethylene (HFO-1132(Z)).

### I. Description of Azeotrope or Azeotrope-Like Compositions

An “azeotrope” composition is a unique combination of two or more components. An azeotrope composition can be characterized in various ways. For example, at a given pressure, an azeotrope composition boils at a constant characteristic temperature which is either greater than the higher boiling point component (maximum boiling azeotrope) or less than the lower boiling point component (minimum boiling azeotrope). At this characteristic temperature the same composition will exist in both the vapor and liquid phases. The azeotrope composition does not fractionate upon boiling or evaporation. Therefore, the components of the azeotrope composition cannot be separated during a phase change.

An azeotrope composition is also characterized in that, at the characteristic azeotrope temperature, the bubble point pressure of the liquid phase is identical to the dew point pressure of the vapor phase.

The behavior of an azeotrope composition is in contrast with that of a non-azeotrope composition in which during boiling or evaporation, the liquid composition changes to a substantial degree.

For the purposes of the present disclosure, an azeotrope composition is characterized as that composition which boils at a constant characteristic temperature, the temperature being lower (a minimum boiling azeotrope) than the boiling points of the two or more components, and thereby having the same composition in both the vapor and liquid phases.

Azeotropes can be classified as being homogeneous or heterogeneous. According to Seader and Henley (Separation Process Principles, Wiley, Second Edition, 2006, pp. 123-126), if only one liquid phase exists, the mixture forms a homogeneous azeotrope; if more than one liquid is present, the azeotrope is heterogeneous. For a fixed temperature, heterogeneous azeotropes, according to Seader and Henley, have total pressures and phase compositions that remain constant across the multiphase region (a region that is sometimes referred to as the “miscibility gap”). In contrast, for a fixed temperature, homogeneous azeotropes yield only one unique total pressure where phase compositions are constant given that a multiphase region is, by definition, absent. While both heterogeneous and homogeneous azeotropes share features of constant composition, the presence or absence of a liquid multiphase region yields a difference that permits or prevents their application, use, and/or otherwise treatment of the resulting azeotrope; therefore, distinguishing an azeotrope as homogeneous or heterogeneous becomes critically important and necessary for their application, use, and/or otherwise treatment.

One of ordinary skill in the art would understand however that at different pressures, both the composition and the boiling point of the azeotrope composition will vary to some extent. Therefore, depending on the temperature and/or pressure, an azeotrope composition can have a variable composition. The skilled person would therefore understand that composition ranges, rather than fixed compositions, can be used to define azeotrope compositions. In addition, an azeotrope may be defined in terms of exact weight percentages of each component of the compositions characterized by a fixed boiling point at a specified pressure.

An “azeotrope-like” composition is a composition of two or more components which behaves substantially as an azeotrope composition. Thus, for the purposes of this disclosure, an azeotrope-like composition is a combination of two or more different components which, when in liquid form under given pressure, will boil at a substantially constant temperature, and which will provide a vapor composition substantially identical to the liquid composition undergoing boiling.

Azeotrope or azeotrope-like compositions can be identified using a number of different methods.

For the purposes of this disclosure the azeotrope or azeotrope-like composition can be identified experimentally using an ebulliometer (Walas, Phase Equilibria in Chemical Engineering, Butterworth-Heinemann, 1985, 533-544). An ebulliometer is designed to provide extremely accurate measurements of the boiling points of liquids by measuring the temperature of the vapor-liquid equilibrium.

The boiling points of each of the components alone are measured at a constant pressure. As the skilled person will appreciate, for a binary azeotrope or azeotrope-like composition, the boiling point of one of the components of the composition is initially measured. The second component of the composition is then added in varying amounts and the boiling point of each of the obtained compositions is measured using the ebulliometer at said constant pressure.

The measured boiling points are plotted against the composition of the tested composition, for example, for a binary azeotrope, the amount of the second component added to the composition, (expressed as either weight % or mole %). The presence of an azeotrope composition can be identified by the observation of a maximum or minimum boiling temperature which is greater or less than the boiling points of any of the components alone.

As the skilled person will appreciate, the identification of the azeotrope or azeotrope-like composition is made by the comparison of the change in the boiling point of the composition on addition of the second component to the first component, relative to the boiling point of the first component. Thus, it is not necessary that the system be calibrated to the reported boiling point of the particular components in order to measure the change in boiling point.

Static Vapor-Liquid Equilibrium Methods are a class of experimental techniques that can also be used to identify the presence of azeotrope and azeotrope-like compositions. One such technique, known as the PTx method, collects measurements of the total saturation pressure (“P”) exerted by mixtures of known compositions (“x”) at fixed temperatures (“T”) and cell volumes. (Walas, Phase Equilibria in Chemical Engineering, Butterworth-Heinemann, 1985, pp. 537). Using data collected from the PTx experiment, as well as pure component properties of constituents of the mixtures, the thermodynamic properties of the mixture can be accurately characterized by fitting the component's interaction parameters in a well-defined thermodynamic equation; one such equation is a Helmholtz Energy Equation of State (HEOS) described by E. W. Lemmon et al. (Generalized Model for the Thermodynamic Properties of Mixtures, International Journal of Thermophysics, Vol. 20, 1999, pp. 825-835)

The presence of an azeotrope and its corresponding composition can be observed by plotting saturation pressure measurements from PTx data and saturation pressures described by the HEOS as a function of composition. For a given temperature (isotherm), the presence of an azeotrope composition is identified by the observation of a maximum or minimum in total pressure that is greater or less than the pure saturation pressures of any of the components alone.

As used herein, the term “consisting essentially of”, with respect to the components of an azeotrope or azeotrope-like composition or mixture, means the composition contains the indicated components in an azeotrope or azeotrope-like ratio, and may contain additional components provided that the additional components do not form new azeotrope or azeotrope-like systems. For example, azeotrope mixtures consisting essentially of two compounds are those that form binary azeotropes, which optionally may include one or more additional components, provided that the additional components do not render the mixture non-azeotropic and do not form an azeotrope with either or both of the compounds (e.g., do not form a ternary or higher azeotrope).

As used herein, the term “about”, when used in connection with recited weight percentages of the components of the present compositions, includes a deviation of ±0.3% from the recited weight percentage.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As previously discussed, for an azeotrope, at the maximum or minimum boiling point, the composition of the vapor phase will be identical to the composition of the liquid phase. The azeotrope-like composition is therefore that composition of components which provides a substantially constant minimum or maximum boiling point at which substantially constant boiling point the composition of the vapor phase will be substantially identical to the composition of the liquid phase.

II. Azeotrope and Azeotrope-Like Compositions of 1,1-difluoropropene (HFO-1252zc) and chlorotrifluoroethylene (CTFE)

The present disclosure provides a homogeneous minimum boiling azeotrope or azeotrope-like composition comprising effective amounts of 1,1-difluoropropene (HFO-1252zc) and chlorotrifluoroethylene (CTFE). The present disclosure particularly provides an azeotrope or azeotrope-like composition consisting essentially of effective amounts of 1,1-difluoropropene (HFO-1252zc) and chlorotrifluoroethylene (CTFE). The present disclosure provides a minimum-boiling, homogenous azeotrope or azeotrope-like composition consisting of effective amounts of 1,1-difluoropropene (HFO-1252zc) and chlorotrifluoroethylene (CTFE).

The azeotrope or azeotrope-like composition may comprise from about 57.1 wt. % to about 72.9 wt. % of 1,1-difluoropropene (HFO-1252zc) and from about 27.1 wt. % to about 42.9 wt. % of chlorotrifluoroethylene (CTFE) at a temperature of about-29.4° C. to about 78.8° C. and a pressure of about 15.6 psia to about 358.8 psia.

The azeotrope or azeotrope-like composition may consist essentially of from about 57.1 wt. % to about 72.9 wt. % of 1,1-difluoropropene (HFO-1252zc) and from about 27.1 wt. % to about 42.9 wt. % of chlorotrifluoroethylene (CTFE) at a temperature of about −29.4° C. to about 78.8° C. and a pressure of about 15.6 psia to about 358.8 psia.

The azeotrope or azeotrope-like composition may consist of from about 57.1 wt. % to about 72.9 wt. % of 1,1-difluoropropene (HFO-1252zc) and from about 27.1 wt. % to about 42.9 wt. % of chlorotrifluoroethylene (CTFE) at a temperature of about-29.4° C. to about 78.8° C. and a pressure of about 15.6 psia to about 358.8 psia.

The azeotrope or azeotrope-like compositions may comprise of from about 0.1 wt. % to about 99.9 wt. % of 1,1-difluoropropene (HFO-1252zc) and from about 0.1 wt. % to about 99.9 wt. % of chlorotrifluoroethylene (CTFE), and more specifically, from about 19.2 wt. % to about 99.9 wt. % of 1,1-difluoropropene (HFO-1252zc) and from about 0.1 wt. % to about 80.8 wt. % of chlorotrifluoroethylene (CTFE), and still more specifically, about 57.5 wt. % of 1,1-difluoropropene (HFO-1252zc) and about 42.5 wt. % of chlorotrifluoroethylene (CTFE) at a pressure of about 358.8 psia.

The azeotrope or azeotrope-like compositions may consist essentially of from about 0.1 wt. % to about 99.9 wt. % of 1,1-difluoropropene (HFO-1252zc) and from about 0.1 wt. % to about 99.9 wt. % of chlorotrifluoroethylene (CTFE), and more specifically from about 19.2 wt. % to about 99.9 wt. % of 1,1-difluoropropene (HFO-1252zc) and from about 0.1 wt. % to about 80.8 wt. % of chlorotrifluoroethylene (CTFE), and still more specifically, about 57.5 wt. % of 1,1-difluoropropene (HFO-1252zc) and about 42.5 wt. % of chlorotrifluoroethylene (CTFE) at a pressure of about 358.8 psia.

The azeotrope or azeotrope-like compositions may consist of from about 0.1 wt. % to about 99.9 wt. % of 1,1-difluoropropene (HFO-1252zc) and from about 0.1 wt. % to about 99.9 wt. % of chlorotrifluoroethylene (CTFE), and more specifically from about 19.2 wt. % to about 99.9 wt. % of 1,1-difluoropropene (HFO-1252zc) and from about 0.1 wt. % to about 80.8 wt. % of chlorotrifluoroethylene (CTFE), and still more specifically, about 57.5 wt. % of 1,1-difluoropropene (HFO-1252zc) and about 42.5 wt. % of chlorotrifluoroethylene (CTFE) at a pressure of about 358.8 psia.

In other words, the compositions may comprise 1,1-difluoropropene (HFO-1252zc) in an amount of as much as about 99.9 wt. %, or about 57.5 wt. %, or as little as about 19.2 wt. %, or about 0.1 wt. %, or by any two of the foregoing values as endpoints, for example from about 0.1 wt. % to about 99.9 wt. %, from about 19.2 wt. % to about 99.9 wt. %, and/or about 57.5 wt. %, based on the total weight of the 1,1-difluoropropene (HFO-1252zc) and chlorotrifluoroethylene (CTFE) of the azeotrope or azeotrope-like composition at a pressure of about 358.8 psia.

In other words, the compositions may consist essentially of 1,1-difluoropropene (HFO-1252zc) in an amount of as much as about 99.9 wt. %, or about 57.5 wt. %, or as little as about 19.2 wt. %, or about 0.1 wt. %, or by any two of the foregoing values as endpoints, for example from about 0.1 wt. % to about 99.9 wt. %, from about 19.2 wt. % to about 99.9 wt. %, and/or about 57.5 wt. %, based on the total weight of the 1,1-difluoropropene (HFO-1252zc) and chlorotrifluoroethylene (CTFE) of the azeotrope or azeotrope-like composition at a pressure of about 358.8 psia.

In other words, the compositions may consist of 1,1-difluoropropene (HFO-1252zc) in an amount of as much as about 99.9 wt. %, or about 57.5 wt. %, or as little as about 19.2 wt. %, or about 0.1 wt. %, or by any two of the foregoing values as endpoints, for example from about 0.1 wt. % to about 99.9 wt. %, from about 19.2 wt. % to about 99.9 wt. %, and/or about 57.5 wt. %, based on the total weight of the 1,1-difluoropropene (HFO-1252zc) and chlorotrifluoroethylene (CTFE) of the azeotrope or azeotrope-like composition at a pressure of about 358.8 psia.

In other words, the compositions may comprise chlorotrifluoroethylene (CTFE) in an amount of as much as about 99.9 wt. %, or about 80.8 wt. %, or about 42.5 wt. %, or as little as about 0.1 wt. %, or by any two of the foregoing values as endpoints, for example from about 0.1 wt. % to about 99.9 wt. %, from about 0.1 wt. % to about 80.8 wt. %, and/or about 42.5 wt. %, based on the total weight of the 1,1-difluoropropene (HFO-1252zc) and chlorotrifluoroethylene (CTFE) of the azeotrope or azeotrope-like composition at a pressure of about 358.8 psia.

In other words, the compositions may consist essentially of chlorotrifluoroethylene (CTFE) in an amount of as much as about 99.9 wt. %, or about 80.8 wt. %, or about 42.5 wt. %, or as little as about 0.1 wt. %, or by any two of the foregoing values as endpoints, for example from about 0.1 wt. % to about 99.9 wt. %, from about 0.1 wt. % to about 80.8 wt. %, and/or about 42.5 wt. %, based on the total weight of the 1,1-difluoropropene (HFO-1252zc) and chlorotrifluoroethylene (CTFE) of the azeotrope or azeotrope-like composition at a pressure of about 358.8 psia.

In other words, the compositions may consist of chlorotrifluoroethylene (CTFE) in an amount of as much as about 99.9 wt. %, or about 80.8 wt. %, or about 42.5 wt. %, or as little as about 0.1 wt. %, or by any two of the foregoing values as endpoints, for example from about 0.1 wt. % to about 99.9 wt. %, from about 0.1 wt. % to about 80.8 wt. %, and/or about 42.5 wt. %, based on the total weight of the 1,1-difluoropropene (HFO-1252zc) and chlorotrifluoroethylene (CTFE) of the azeotrope or azeotrope-like composition at a pressure of about 358.8 psia.

The compositions may have azeotropic or azeotrope-like characteristics at a temperature of about 78.8° C., about 68.9° C., about 59.0° C., about 49.3° C., about 39.5° C., about 29.5° C., about 19.7° C., about 9.9° C., about 0.3° C., about −9.3° C., about −19.1° C., about −29.4° C., or within any range encompassed by any two of the foregoing values as endpoints, for example from about −29.4° C. to about 78.8° C.

The compositions may have azeotropic or azeotrope-like characteristics at a pressure of about 358.8 psia, about 292.3 psia, about 235.9 psia, about 188.7 psia, about 148.6 psia, about 114.8 psia, about 87.2 psia, about 65.2 psia, about 47.9 psia, about 34.4 psia, about 23.8 psia, about 15.6 psia, or within any range encompassed by any two of the foregoing values as endpoints, for example from about 15.6 psia to about 358.8 psia, from about 15.6 psia to about 188.7 psia, from about 23.8 psia to about 235.9 psia, from about 34.4 psia to about 358.8 psia, or from about 47.9 psia to about 358.8 psia.

Specifically, and as described in Table 1 below, the azeotrope or azeotrope-like composition of 1,1-difluoropropene (HFO-1252zc) and chlorotrifluoroethylene (CTFE) may correlate with pressure (psia) and saturation temperature (° C.).

In column (i) of Table 1 below, azeotrope-like composition ranges may depend as based upon the method used to determine such range. Specifically, (i) temperature glide is the difference between the saturated vapor temperature and the saturated liquid temperature at a fixed pressure in thermodynamic equilibrium. Consequently, an azeotrope composition may have a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This method was used to determine the relative compositions in column (i) of Table 1 below, which may be regarded as the broadest azeotrope-like composition range.

In column (ii) of Table 1 below, the relative volatility is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This may be regarded as an intermediate azeotrope-like composition range.

In column (iii) of Table 1 below a relative volatility of 1.05 is substantially close to 1.0 and therefore, compositions that satisfy such relative volatility are considered azeotrope-like. This may be regarded as the narrowest azeotrope-like composition range.

Column (1) of Table 1 below describes the azeotrope composition that, at a given pressure, the azeotrope composition boils at a constant characteristic temperature which is less than the lower boiling point component (minimum boiling azeotrope). At this characteristic temperature the same composition will exist in both the vapor and liquid phases. Therefore, the components of the azeotrope composition cannot be separated during a phase change and are regarded as the azeotrope composition. Such compositional values are presented in Column (1) of Table 1 below corresponding to each pressure and saturation temperature.

According, and in view of the foregoing, the azeotrope or azeotrope-like composition of 1,1-difluoropropene (HFO-1252zc) and chlorotrifluoroethylene (CTFE) may comprise any of the values as described in each of columns (1), (i), (ii) or (iii) in each row of Table 1 below.

The azeotrope or azeotrope-like composition of 1,1-difluoropropene (HFO-1252zc) and chlorotrifluoroethylene (CTFE) may consist essentially of any of the values as described in each of columns (1), (i), (ii) or (iii) in each row of Table 1 below.

The azeotrope or azeotrope-like composition of 1,1-difluoropropene (HFO-1252zc) and chlorotrifluoroethylene (CTFE) may consist of any of the values described in each of columns (1), (i), (ii) or (iii) in each row of Table 1 below.

TABLE 1
(1) Azeotrope Composition and (2) Azeotrope-like composition ranges of
1,1-difluoropropene (HFO-1252zc) and chlorotrifluoroethylene (CTFE)

| | | (1) Azeotropic | | (2) Azeotropic-Like Composition Range | | | | | |
|---|---|---|---|---|---|---|---|---|---|
| Sat. | Sat. | Composition | | (i) Glide <0.5° C. | | (ii) Rel. Vol. <1.1 | | (iii) Rel. Vol. <1.05 | |
| Press. | Temp. | Wt. % | Wt. % | Wt. % | Wt. % | Wt. % | Wt. % | Wt. % | Wt. % |
| (psia) | (° C.) | HFO-1252zc | CTFE | HFO-1252zc | CTFE | HFO-1252zc | CTFE | HFO-1252zc | CTFE |
| 15.6 | −29.4 | 72.9 | 27.1 | 0.1 to 99.9 | 0.1 to 99.9 | 36.0 to 99.9 | 0.1 to 64.0 | 50.4 to 99.9 | 0.1 to 49.6 |
| 23.8 | −19.1 | 68.5 | 31.5 | 0.1 to 99.9 | 0.1 to 99.9 | 32.2 to 99.9 | 0.1 to 67.8 | 46.4 to 99.9 | 0.1 to 53.6 |
| 34.4 | −9.3 | 65.3 | 34.7 | 0.1 to 99.9 | 0.1 to 99.9 | 28.9 to 99.9 | 0.1 to 71.1 | 43.1 to 99.9 | 0.1 to 56.9 |
| 47.9 | 0.3 | 62.8 | 37.2 | 0.1 to 99.9 | 0.1 to 99.9 | 25.9 to 99.9 | 0.1 to 74.1 | 40.4 to 99.9 | 0.1 to 59.6 |
| 65.2 | 9.9 | 60.9 | 39.1 | 0.1 to 99.9 | 0.1 to 99.9 | 23.2 to 99.9 | 0.1 to 76.8 | 37.9 to 99.9 | 0.1 to 62.1 |
| 87.2 | 19.7 | 59.5 | 40.5 | 0.1 to 99.9 | 0.1 to 99.9 | 20.4 to 99.9 | 0.1 to 79.6 | 35.6 to 99.9 | 0.1 to 64.4 |
| 114.8 | 29.5 | 58.4 | 41.6 | 0.1 to 99.9 | 0.1 to 99.9 | 17.7 to 99.9 | 0.1 to 82.3 | 33.3 to 99.9 | 0.1 to 66.7 |
| 148.6 | 39.5 | 57.7 | 42.3 | 0.1 to 99.9 | 0.1 to 99.9 | 14.8 to 99.9 | 0.1 to 85.2 | 31.0 to 99.9 | 0.1 to 69.0 |
| 188.7 | 49.3 | 57.3 | 42.7 | 0.1 to 99.9 | 0.1 to 99.9 | 11.8 to 99.9 | 0.1 to 88.2 | 28.8 to 99.9 | 0.1 to 71.2 |
| 235.9 | 59 | 57.1 | 42.9 | 0.1 to 99.9 | 0.1 to 99.9 | 8.4 to 99.9 | 0.1 to 91.6 | 36.3 to 99.9 | 0.1 to 63.7 |
| 292.3 | 68.9 | 57.2 | 42.8 | 0.1 to 99.9 | 0.1 to 99.9 | 4.2 to 99.9 | 0.1 to 95.8 | 23.3 to 99.9 | 0.1 to 76.7 |
| 358.8 | 78.8 | 57.5 | 42.5 | 0.1 to 99.9 | 0.1 to 99.9 | 0.1 to 99.9 | 0.1 to 99.9 | 19.2 to 99.9 | 0.1 to 80.8 |

III. Azeotrope and Azeotrope-Like Compositions of 1,1-difluoropropene (Hfo-1252zc) and hexafluoropropylene (Hfp)

The present disclosure provides a homogeneous minimum boiling azeotrope or azeotrope-like composition comprising effective amounts of 1,1-difluoropropene (HFO-1252zc) and hexafluoropropylene (HFP). The present disclosure particularly provides an azeotrope or azeotrope-like composition consisting essentially of effective amounts of 1,1-difluoropropene (HFO-1252zc) and hexafluoropropylene (HFP). The present disclosure provides a minimum-boiling, homogenous azeotrope or azeotrope-like composition consisting of effective amounts of 1,1-difluoropropene (HFO-1252zc) and hexafluoropropylene (HFP).

The azeotrope or azeotrope-like composition may comprise from about 23.9 wt. % to about 40.3 wt. % of 1,1-difluoropropene (HFO-1252zc) and from about 59.7 wt. % to about 76.1 wt. % of hexafluoropropylene (HFP) at a temperature of about −29.3° C. to about 78.7° C. and a pressure of about 17.4 psia to about 404.1 psia.

The azeotrope or azeotrope-like composition may consist essentially of from about 23.9 wt. % to about 40.3 wt. % of 1,1-difluoropropene (HFO-1252zc) and from about 59.7 wt. % to about 76.1 wt. % of hexafluoropropylene (HFP) at a temperature of about −29.3° C. to about 78.7° C. and a pressure of about 17.4 psia to about 404.1 psia.

The azeotrope or azeotrope-like composition may consist of from about 23.9 wt. % to about 40.3 wt. % of 1,1-difluoropropene (HFO-1252zc) and from about 59.7 wt. % to about 76.1 wt. % of hexafluoropropylene (HFP) at a temperature of about −29.3° C. to about 78.7° C. and a pressure of about 17.4 psia to about 404.1 psia.

The azeotrope or azeotrope-like compositions may comprise of from about 0.1 wt. % to about 99.9 wt. % of 1,1-difluoropropene (HFO-1252zc) and from about 0.1 wt. % to about 99.9 wt. % of hexafluoropropylene (HFP), and more specifically, from about 0.1 wt. % to about 57.5 wt. % of 1,1-difluoropropene (HFO-1252zc) and from about 42.5 wt. % to about 99.9 wt. % of hexafluoropropylene (HFP), from about 0.1 wt. % to about 41.7 wt. % of 1,1-difluoropropene (HFO-1252zc) and from about 58.3 wt. % to about 99.9 wt. % of hexafluoropropylene (HFP), and still more specifically, about 23.9 wt. % of 1,1-difluoropropene (HFO-1252zc) and about 76.1 wt. % of hexafluoropropylene (HFP) at a pressure of about 404.1 psia.

The azeotrope or azeotrope-like compositions may consist essentially of from about 0.1 wt. % to about 99.9 wt. % of 1,1-difluoropropene (HFO-1252zc) and from about 0.1 wt. % to about 99.9 wt. % of hexafluoropropylene (HFP), and more specifically, from about 0.1 wt. % to about 57.5 wt. % of 1,1-difluoropropene (HFO-1252zc) and from about 42.5 wt. % to about 99.9 wt. % of hexafluoropropylene (HFP), from about 0.1 wt. % to about 41.7 wt. % of 1,1-difluoropropene (HFO-1252zc) and from about 58.3 wt. % to about 99.9 wt. % of hexafluoropropylene (HFP), and still more specifically, about 23.9 wt. % of 1,1-difluoropropene (HFO-1252zc) and about 76.1 wt. % of hexafluoropropylene (HFP) at a pressure of about 404.1 psia.

The azeotrope or azeotrope-like compositions may consist of from about 0.1 wt. % to about 99.9 wt. % of 1,1-difluoropropene (HFO-1252zc) and from about 0.1 wt. % to about 99.9 wt. % of hexafluoropropylene (HFP), and more specifically, from about 0.1 wt. % to about 57.5 wt. % of 1,1-difluoropropene (HFO-1252zc) and from about 42.5 wt. % to about 99.9 wt. % of hexafluoropropylene (HFP), from about 0.1 wt. % to about 41.7 wt. % of 1,1-difluoropropene (HFO-1252zc) and from about 58.3 wt. % to about 99.9 wt. % of hexafluoropropylene (HFP), and still more specifically, about 23.9 wt. % of 1,1-difluoropropene (HFO-1252zc) and about 76.1 wt. % of hexafluoropropylene (HFP) at a pressure of about 404.1 psia.

In other words, the compositions may comprise 1,1-difluoropropene (HFO-1252zc) in an amount of as much as about 99.9 wt. %, or about 57.5 wt. %, or about 41.7 wt. %, or about 23.9 wt. %, or as little as about 0.1 wt. %, or by any two of the foregoing values as endpoints, for example from about 0.1 wt. % to about 99.9 wt. %, from about 0.1 wt. % to about 57.5 wt. %, from about 0.1 wt. % to about 41.7 wt. %, and/or about 23.9 wt. %, based on the total weight of the 1,1-difluoropropene (HFO-1252zc) and hexafluoropropylene (HFP) of the azeotrope or azeotrope-like composition at a pressure of about 404.1 psia.

In other words, the compositions may consist essentially of 1,1-difluoropropene (HFO-1252zc) in an amount of as much as about 99.9 wt. %, or about 57.5 wt. %, or about 41.7 wt. %, or about 23.9 wt. %, or as little as about 0.1 wt. %, or by any two of the foregoing values as endpoints, for example from about 0.1 wt. % to about 99.9 wt. %, from about 0.1 wt. % to about 57.5 wt. %, from about 0.1 wt. % to about 41.7 wt. %, and/or about 23.9 wt. %, based on the total weight of the 1,1-difluoropropene (HFO-1252zc) and hexafluoropropylene (HFP) of the azeotrope or azeotrope-like composition at a pressure of about 404.1 psia.

In other words, the compositions may consist of 1,1-difluoropropene (HFO-1252zc) in an amount of as much as about 99.9 wt. %, or about 57.5 wt. %, or about 41.7 wt. %, or about 23.9 wt. %, or as little as about 0.1 wt. %, or by any two of the foregoing values as endpoints, for example from about 0.1 wt. % to about 99.9 wt. %, from about 0.1 wt. % to about 57.5 wt. %, from about 0.1 wt. % to about 41.7 wt. %, and/or about 23.9 wt. %, based on the total weight of the 1,1-difluoropropene (HFO-1252zc) and hexafluoropropylene (HFP) of the azeotrope or azeotrope-like composition at a pressure of about 404.1 psia.

In other words, the compositions may comprise hexafluoropropylene (HFP) in an amount of as much as about 99.9 wt. %, or about 76.1 wt. %, or about 58.3 wt. %, or about 42.5 wt. %, or as little as about 0.1 wt. %, or by any two of the foregoing values as endpoints, for example from about 0.1 wt. % to about 99.9 wt. %, from about 42.5 wt. % to about 99.9 wt. %, from about 58.3 wt. % to about 99.9 wt. %, and/or about 76.1 wt. %, based on the total weight of the 1,1-difluoropropene (HFO-1252zc) and hexafluoropropylene (HFP) of the azeotrope or azeotrope-like composition at a pressure of about 404.1 psia.

In other words, the compositions may consist essentially of hexafluoropropylene (HFP) in an amount of as much as about 99.9 wt. %, or about 76.1 wt. %, or about 58.3 wt. %, or about 42.5 wt. %, or as little as about 0.1 wt. %, or by any two of the foregoing values as endpoints, for example from about 0.1 wt. % to about 99.9 wt. %, from about 42.5 wt. % to about 99.9 wt. %, from about 58.3 wt. % to about 99.9 wt. %, and/or about 76.1 wt. %, based on the total weight of the 1,1-difluoropropene (HFO-1252zc) and hexafluoropropylene (HFP) of the azeotrope or azeotrope-like composition at a pressure of about 404.1 psia.

In other words, the compositions may consist of hexafluoropropylene (HFP) in an amount of as much as about 99.9 wt. %, or about 76.1 wt. %, or about 58.3 wt. %, or about 42.5 wt. %, or as little as about 0.1 wt. %, or by any two of the foregoing values as endpoints, for example from about 0.1 wt. % to about 99.9 wt. %, from about 42.5 wt. % to about 99.9 wt. %, from about 58.3 wt. % to about 99.9 wt. %, and/or about 76.1 wt. %, based on the total weight of the 1,1-difluoropropene (HFO-1252zc) and hexafluoropropylene (HFP) of the azeotrope or azeotrope-like composition at a pressure of about 404.1 psia.

The compositions may have azeotropic or azeotrope-like characteristics at a temperature of about 78.7° C., about 68.8° C., about 58.9° C., about 49.2° C., about 39.4° C., about 29.4° C., about 19.5° C., about 10.1° C., about 0.4° C., about −9.3° C., about −19.2° C., about −29.3° C., or within any range encompassed by any two of the foregoing values as endpoints, for example from about −29.3° C. to about 78.7° C.

The compositions may have azeotropic or azeotrope-like characteristics at a pressure of about 404.1 psia, about 327.9 psia, about 263.7 psia, about 210.7 psia, about 165.4 psia, about 127.6 psia, about 96.9 psia, about 73.2 psia, about 53.6 psia, about 38.2 psia, about 26.4 psia, about 17.4 psia, or within any range encompassed by any two of the foregoing values as endpoints, for example from about 17.4 psia to about 404.1 psia, from about 17.4 psia to about 210.7 psia, from about 26.4 psia to about 263.7 psia, from about 38.2 psia to about 404.1 psia, or from about 53.6 psia to about 404.1 psia.

Specifically, and as described in Table 1 below, the azeotrope or azeotrope-like composition of 1,1-difluoropropene (HFO-1252zc) and hexafluoropropylene (HFP) may correlate with pressure (psia) and saturation temperature (C).

In column (i) of Table 2 below, azeotrope-like composition ranges may depend as based upon the method used to determine such range. Specifically, (i) temperature glide is the difference between the saturated vapor temperature and the saturated liquid temperature at a fixed pressure in thermodynamic equilibrium. Consequently, an azeotrope composition may have a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This method was used to determine the relative compositions in column (i) of Table 1 below, which may be regarded as the broadest azeotrope-like composition range.

In column (ii) of Table 2 below, the relative volatility is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This may be regarded as an intermediate azeotrope-like composition range.

In column (iii) of Table 2 below a relative volatility of 1.05 is substantially close to 1.0 and therefore, compositions that satisfy such relative volatility are considered azeotrope-like. This may be regarded as the narrowest azeotrope-like composition range.

Column (1) of Table 2 below describes the azeotrope composition that, at a given pressure, the azeotrope composition boils at a constant characteristic temperature which is less than the lower boiling point component (minimum boiling azeotrope). At this characteristic temperature the same composition will exist in both the vapor and liquid phases. Therefore, the components of the azeotrope composition cannot be separated during a phase change and are regarded as the azeotrope composition. Such compositional values are presented in Column (1) of Table 2 below corresponding to each pressure and saturation temperature.

According, and in view of the foregoing, the azeotrope or azeotrope-like composition of 1,1-difluoropropene (HFO-1252zc) and hexafluoropropylene (HFP) may comprise any of the values as described in each of columns (1), (i), (ii) or (iii) in each row of Table 2 below.

The azeotrope or azeotrope-like composition of 1,1-difluoropropene (HFO-1252zc) and hexafluoropropylene (HFP) may consist essentially of any of the values as described in each of columns (1), (i), (ii) or (iii) in each row of Table 2 below.

The azeotrope or azeotrope-like composition of 1,1-difluoropropene (HFO-1252zc) and hexafluoropropylene (HFP) may consist of any of the values described in each of columns (1), (i), (ii) or (iii) in each row of Table 2 below.

TABLE 2
(1) Azeotrope Composition and (2) Azeotrope-like composition ranges
of 1,1-difluoropropene (HFO-1252zc) and hexafluoropropylene (HFP)

| | | (1) Azeotropic | | (2) Azeotropic-Like Composition Range | | | | | |
|---|---|---|---|---|---|---|---|---|---|
| Sat. | Sat. | Composition | | (i) Glide <0.5° C. | | (ii) Rel. Vol. <1.1 | | (iii) Rel. Vol. <1.05 | |
| Press. | Temp. | Wt. % | Wt. % | Wt. % | Wt. % | Wt. % | Wt. % | Wt. % | Wt. % |
| (psia) | (° C.) | HFP | HFO-1252zc | HFP | HFO-1252zc | HFP | HFO-1252zc | HFP | HFO-1252zc |
| 17.4 | −29.3 | 59.7 | 40.3 | 0.1 to 99.9 | 0.1 to 99.9 | 49.8 to 69.5 | 30.5 to 50.2 | 54.7 to 64.7 | 35.3 to 45.3 |
| 26.4 | −19.2 | 61.6 | 38.4 | 0.1 to 99.9 | 0.1 to 99.9 | 51.0 to 72.2 | 27.8 to 49.0 | 56.3 to 67.0 | 33.0 to 43.7 |
| 38.2 | −9.3 | 63.4 | 36.6 | 0.1 to 99.9 | 0.1 to 99.9 | 51.9 to 74.8 | 25.2 to 48.1 | 57.6 to 69.1 | 30.9 to 42.4 |
| 53.6 | 0.4 | 64.9 | 35.1 | 0.1 to 99.9 | 0.1 to 99.9 | 52.6 to 77.4 | 22.6 to 47.4 | 58.6 to 71.2 | 28.8 to 41.4 |
| 73.2 | 10.1 | 66.4 | 33.6 | 0.1 to 99.9 | 0.1 to 99.9 | 53.1 to 80.0 | 20.0 to 46.9 | 59.6 to 73.2 | 26.8 to 40.4 |
| 96.9 | 19.5 | 67.7 | 32.3 | 0.1 to 99.9 | 0.1 to 99.9 | 53.3 to 82.7 | 17.3 to 46.7 | 60.4 to 75.1 | 24.9 to 39.6 |
| 127.6 | 29.4 | 69 | 31 | 0.1 to 99.9 | 0.1 to 99.9 | 53.3 to 85.9 | 14.1 to 46.7 | 61.0 to 77.3 | 22.7 to 39.0 |
| 165.4 | 39.4 | 70.3 | 29.7 | 0.1 to 99.9 | 0.1 to 99.9 | 52.9 to 89.9 | 10.1 to 47.1 | 61.4 to 79.6 | 20.4 to 38.6 |
| 210.7 | 49.2 | 71.6 | 28.4 | 0.1 to 99.9 | 0.1 to 99.9 | 52.1 to 95.4 | 4.6 to 47.9 | 61.6 to 82.3 | 17.7 to 38.4 |
| 263.7 | 58.9 | 72.9 | 27.1 | 0.1 to 99.9 | 0.1 to 99.9 | 50.7 to 99.9 | 0.1 to 49.3 | 61.5 to 85.6 | 14.4 to 38.5 |
| 327.9 | 68.8 | 74.3 | 25.7 | 0.1 to 99.9 | 0.1 to 99.9 | 48.0 to 99.9 | 0.1 to 52.0 | 60.7 to 91.0 | 9.0 to 39.3 |
| 404.1 | 78.7 | 76.1 | 23.9 | 0.1 to 99.9 | 0.1 to 99.9 | 42.5 to 99.9 | 0.1 to 57.5 | 58.3 to 99.9 | 0.1 to 41.7 |

IV. Azeotrope and Azeotrope-Like Compositions of 1,1-difluoropropene (HFO-1252zc) and cis-1,2-difluoroethylene (HFO-1132(Z))

The present disclosure provides an azeotrope or azeotrope-like composition comprising effective amounts of 1,1-difluoropropene (HFO-1252zc) and cis-1,2-difluoroethylene (HFO-1132(Z)). The present disclosure particularly provides an azeotrope or azeotrope-like composition consisting essentially of effective amounts of 1,1-difluoropropene (HFO-1252zc) and cis-1,2-difluoroethylene (HFO-1132(Z)). The present disclosure provides a minimum-boiling, homogenous azeotrope or azeotrope-like composition consisting of effective amounts of 1,1-difluoropropene (HFO-1252zc) and cis-1,2-difluoroethylene (HFO-1132(Z)).

The azeotrope or azeotrope-like composition may comprise from about 56.1 wt. % to about 71.0 wt. % of 1,1-difluoropropene (HFO-1252zc) and from about 29.0 wt. % to about 43.9 wt. % of cis-1,2-difluoroethylene (HFO-1132(Z)) at a temperature of about −29.2° C. to about 78.7° C. and a pressure of about 16.7 psia to about 388.2 psia.

The azeotrope or azeotrope-like composition may consist essentially of from about 56.1 wt. % to about 71.0 wt. % of 1,1-difluoropropene (HFO-1252zc) and from about 29.0 wt. % to about 43.9 wt. % of cis-1,2-difluoroethylene (HFO-1132(Z)) at a temperature of about −29.2° C. to about 78.7° C. and a pressure of about 16.7 psia to about 388.2 psia.

The azeotrope or azeotrope-like composition may consist of from about 56.1 wt. % to about 71.0 wt. % of 1,1-difluoropropene (HFO-1252zc) and from about 29.0 wt. % to about 43.9 wt. % of cis-1,2-difluoroethylene (HFO-1132(Z)) at a temperature of about −29.2° C. to about 78.7° C. and a pressure of about 16.7 psia to about 388.2 psia.

The azeotrope or azeotrope-like compositions may comprise of from about 0.1 wt. % to about 99.9 wt. % of 1,1-difluoropropene (HFO-1252zc) and from about 0.1 wt. % to about 99.9 wt. % of cis-1,2-difluoroethylene (HFO-1132(Z)), and more specifically, from about 34.4 wt. % to about 79.4 wt. % of 1,1-difluoropropene (HFO-1252zc) and from about 20.6 wt. % to about 65.6 wt. % of cis-1,2-difluoroethylene (HFO-1132(Z)), from about 44.9 wt. % to about 67.6 wt. % of 1,1-difluoropropene (HFO-1252zc) and from about 32.4 wt. % to about 55.1 wt. % of cis-1,2-difluoroethylene (HFO-1132(Z)), and still more specifically, about 56.1 wt. % of 1,1-difluoropropene (HFO-1252zc) and about 43.9 wt. % of cis-1,2-difluoroethylene (HFO-1132(Z)) at a pressure of about 388.2 psia.

The azeotrope or azeotrope-like compositions may consist essentially of from about 0.1 wt. % to about 99.9 wt. % of 1,1-difluoropropene (HFO-1252zc) and from about 0.1 wt. % to about 99.9 wt. % of cis-1,2-difluoroethylene (HFO-1132(Z)), and more specifically, from about 34.4 wt. % to about 79.4 wt. % of 1,1-difluoropropene (HFO-1252zc) and from about 20.6 wt. % to about 65.6 wt. % of cis-1,2-difluoroethylene (HFO-1132(Z)), from about 44.9 wt. % to about 67.6 wt. % of 1,1-difluoropropene (HFO-1252zc) and from about 32.4 wt. % to about 55.1 wt. % of cis-1,2-difluoroethylene (HFO-1132(Z)), and still more specifically, about 56.1 wt. % of 1,1-difluoropropene (HFO-1252zc) and about 43.9 wt. % of cis-1,2-difluoroethylene (HFO-1132(Z)) at a pressure of about 388.2 psia.

The azeotrope or azeotrope-like compositions may consist of from about 0.1 wt. % to about 99.9 wt. % of 1,1-difluoropropene (HFO-1252zc) and from about 0.1 wt. % to about 99.9 wt. % of cis-1,2-difluoroethylene (HFO-1132(Z)), and more specifically, from about 34.4 wt. % to about 79.4 wt. % of 1,1-difluoropropene (HFO-1252zc) and from about 20.6 wt. % to about 65.6 wt. % of cis-1,2-difluoroethylene (HFO-1132(Z)), from about 44.9 wt. % to about 67.6 wt. % of 1,1-difluoropropene (HFO-1252zc) and from about 32.4 wt. % to about 55.1 wt. % of cis-1,2-difluoroethylene (HFO-1132(Z)), and still more specifically, about 56.1 wt. % of 1,1-difluoropropene (HFO-1252zc) and about 43.9 wt. % of cis-1,2-difluoroethylene (HFO-1132(Z)) at a pressure of about 388.2 psia.

In other words, the compositions may comprise 1,1-difluoropropene (HFO-1252zc) in an amount of as much as about 99.9 wt. %, or about 79.4 wt. %, or about 67.6 wt. %, or about 56.1 wt. %, or as little as about 44.9 wt. %, or about 34.4 wt. %, or about 0.1 wt. %, or by any two of the foregoing values as endpoints, for example from about 0.1 wt. % to about 99.9 wt. %, from about 34.4 wt. % to about 79.4 wt. %, from about 44.9 wt. % to about 67.6 wt. %, and/or about 56.1 wt. %, based on the total weight of the 1,1-difluoropropene (HFO-1252zc) and cis-1,2-difluoroethylene (HFO-1132(Z)) of the azeotrope or azeotrope-like composition at a pressure of about 388.2 psia.

In other words, the compositions may consist essentially of 1,1-difluoropropene (HFO-1252zc) in an amount of as much as about 99.9 wt. %, or about 79.4 wt. %, or about 67.6 wt. %, or about 56.1 wt. %, or as little as about 44.9 wt. %, or about 34.4 wt. %, or about 0.1 wt. %, or by any two of the foregoing values as endpoints, for example from about 0.1 wt. % to about 99.9 wt. %, from about 34.4 wt. % to about 79.4 wt. %, from about 44.9 wt. % to about 67.6 wt. %, and/or about 56.1 wt. %, based on the total weight of the 1,1-difluoropropene (HFO-1252zc) and cis-1,2-difluoroethylene (HFO-1132(Z)) of the azeotrope or azeotrope-like composition at a pressure of about 388.2 psia.

In other words, the compositions may consist of 1,1-difluoropropene (HFO-1252zc) in an amount of as much as about 99.9 wt. %, or about 79.4 wt. %, or about 67.6 wt. %, or about 56.1 wt. %, or as little as about 44.9 wt. %, or about 34.4 wt. %, or about 0.1 wt. %, or by any two of the foregoing values as endpoints, for example from about 0.1 wt. % to about 99.9 wt. %, from about 34.4 wt. % to about 79.4 wt. %, from about 44.9 wt. % to about 67.6 wt. %, and/or about 56.1 wt. %, based on the total weight of the 1,1-difluoropropene (HFO-1252zc) and cis-1,2-difluoroethylene (HFO-1132(Z)) of the azeotrope or azeotrope-like composition at a pressure of about 388.2 psia.

In other words, the compositions may comprise cis-1,2-difluoroethylene (HFO-1132(Z)) in an amount of as much as about 99.9 wt. %, or about 65.6 wt. %, or about 55.1 wt. %, or about 43.9 wt. %, or as little as about 32.4 wt. %, or about 20.6 wt. %, or about 0.1 wt. %, or by any two of the foregoing values as endpoints, for example from about 0.1 wt. % to about 99.9 wt. %, from about 20.6 wt. % to about 65.6 wt. %, from about 32.4 wt. % to about 55.1 wt. %, and/or about 43.9 wt. %, based on the total weight of the 1,1-difluoropropene (HFO-1252zc) and cis-1,2-difluoroethylene (HFO-1132(Z)) of the azeotrope or azeotrope-like composition at a pressure of about 388.2 psia.

In other words, the compositions may consist essentially of cis-1,2-difluoroethylene (HFO-1132(Z)) in an amount of as much as about 99.9 wt. %, or about 65.6 wt. %, or about 55.1 wt. %, or about 43.9 wt. %, or as little as about 32.4 wt. %, or about 20.6 wt. %, or about 0.1 wt. %, or by any two of the foregoing values as endpoints, for example from about 0.1 wt. % to about 99.9 wt. %, from about 20.6 wt. % to about 65.6 wt. %, from about 32.4 wt. % to about 55.1 wt. %, and/or about 43.9 wt. %, based on the total weight of the 1,1-difluoropropene (HFO-1252zc) and cis-1,2-difluoroethylene (HFO-1132(Z)) of the azeotrope or azeotrope-like composition at a pressure of about 388.2 psia.

In other words, the compositions may consist of cis-1,2-difluoroethylene (HFO-1132(Z)) in an amount of as much as about 99.9 wt. %, or about 65.6 wt. %, or about 55.1 wt. %, or about 43.9 wt. %, or as little as about 32.4 wt. %, or about 20.6 wt. %, or about 0.1 wt. %, or by any two of the foregoing values as endpoints, for example from about 0.1 wt. % to about 99.9 wt. %, from about 20.6 wt. % to about 65.6 wt. %, from about 32.4 wt. % to about 55.1 wt. %, and/or about 43.9 wt. %, based on the total weight of the 1,1-difluoropropene (HFO-1252zc) and cis-1,2-difluoroethylene (HFO-1132(Z)) of the azeotrope or azeotrope-like composition at a pressure of about 388.2 psia.

The compositions may have azeotropic or azeotrope-like characteristics at a temperature of about 78.7° C., about 68.9° C., about 59.0° C., about 49.3° C., about 39.5° C., about 29.5° C., about 19.6° C., about 9.9° C., about 0.2° C., about −9.4° C., about −19.2° C., about −29.2° C., or within any range encompassed by any two of the foregoing values as endpoints, for example from about −29.2° C. to about 78.7° C.

The compositions may have azeotropic or azeotrope-like characteristics at a pressure of about 388.2 psia, about 316.1 psia, about 254.4 psia, about 203.2 psia, about 159.6 psia, about 123.0 psia, about 93.3 psia, about 69.7 psia, about 51.0 psia, about 36.5 psia, about 25.2 psia, about 16.7 psia, or within any range encompassed by any two of the foregoing values as endpoints, for example from about 16.7 psia to about 388.2 psia, from about 16.7 psia to about 203.2 psia, from about 25.2 psia to about 254.4 psia, from about 36.5 psia to about 388.2 psia, or from about 51.0 psia to about 388.2 psia.

Specifically, and as described in Table 3 below, the azeotrope or azeotrope-like composition of 1,1-difluoropropene (HFO-1252zc) and cis-1,2-difluoroethylene (HFO-1132(Z) may correlate with pressure (psia) and saturation temperature (° C.).

In column (i) of Table 3 below, azeotrope-like composition ranges may depend as based upon the method used to determine such range. Specifically, (i) temperature glide is the difference between the saturated vapor temperature and the saturated liquid temperature at a fixed pressure in thermodynamic equilibrium. Consequently, an azeotrope composition may have a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This method was used to determine the relative compositions in column (i) of Table 1 below, which may be regarded as the broadest azeotrope-like composition range.

In column (ii) of Table 3 below, the relative volatility is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This may be regarded as an intermediate azeotrope-like composition range.

In column (iii) of Table 3 below a relative volatility of 1.05 is substantially close to 1.0 and therefore, compositions that satisfy such relative volatility are considered azeotrope-like. This may be regarded as the narrowest azeotrope-like composition range.

Column (1) of Table 3 below describes the azeotrope composition that, at a given pressure, the azeotrope composition boils at a constant characteristic temperature which is less than the lower boiling point component (minimum boiling azeotrope). At this characteristic temperature the same composition will exist in both the vapor and liquid phases. Therefore, the components of the azeotrope composition cannot be separated during a phase change and are regarded as the azeotrope composition. Such compositional values are presented in Column (1) of Table 3 below corresponding to each pressure and saturation temperature.

According, and in view of the foregoing, the azeotrope or azeotrope-like composition of 1,1-difluoropropene (HFO-1252zc) and cis-1,2-difluoroethylene (HFO-1132(Z)) may comprise any of the values as described in each of columns (1), (i), (ii) or (iii) in each row of Table 3 below.

The azeotrope or azeotrope-like composition of 1,1-difluoropropene (HFO-1252zc) and cis-1,2-difluoroethylene (HFO-1132(Z) may consist essentially of any of the values as described in each of columns (1), (i), (ii) or (iii) in each row of Table 3 below.

The azeotrope or azeotrope-like composition of 1,1-difluoropropene (HFO-1252zc) and cis-1,2-difluoroethylene (HFO-1132(Z) may consist of any of the values described in each of columns (1), (i), (ii) or (iii) in each row of Table 3 below.

TABLE 3
(1) Azeotrope Composition and (2) Azeotrope-like composition ranges of 1,1-
difluoropropene (HFO-1252zc) and cis-1,2-difluoroethylene (HFO-1132(Z))

| | | | | (2) Azeotropic-Like Composition Range | | | | | |
|---|---|---|---|---|---|---|---|---|---|
| Sat. | Sat. | (1) Azeotropic Composition | | (i) Glide <0.5° C. | | (ii) Rel. Vol. <1.1 | | (iii) Rel. Vol. <1.05 | |
| Press. | Temp. | Wt. % | Wt. % | Wt. % | Wt. % | Wt. % | Wt. % | Wt. % | Wt. % |
| (psia) | (° C.) | HFO-1252zc | HFO-1132(Z) | HFO-1252zc | HFO-1132(Z) | HFO-1252zc | HFO-1132(Z) | HFO-1252zc | HFO-1132(Z) |
| 16.7 | −29.2 | 71.0 | 29.0 | 49.6 to 99.9 | 0.1 to 50.4 | 63.3 to 78.7 | 21.3 to 36.7 | 67.1 to 74.9 | 25.1 to 32.9 |
| 25.2 | −19.2 | 69.4 | 30.6 | 47.1 to 99.9 | 0.1 to 52.9 | 61.1 to 77.7 | 22.3 to 38.9 | 65.2 to 73.6 | 26.4 to 34.8 |
| 36.5 | −9.4 | 67.8 | 32.2 | 44.5 to 99.9 | 0.1 to 55.5 | 58.9 to 76.8 | 23.2 to 41.1 | 63.3 to 72.4 | 27.6 to 36.7 |
| 51.0 | 0.2 | 66.3 | 33.7 | 41.8 to 99.9 | 0.1 to 58.2 | 56.7 to 76.0 | 24.0 to 43.3 | 61.4 to 71.3 | 28.7 to 38.6 |
| 69.7 | 9.9 | 64.9 | 35.1 | 39.0 to 99.9 | 0.1 to 61.0 | 54.4 to 75.3 | 24.7 to 45.6 | 59.5 to 70.2 | 29.8 to 40.5 |
| 93.3 | 19.6 | 63.4 | 36.6 | 35.9 to 99.9 | 0.1 to 64.1 | 52.1 to 74.8 | 25.2 to 47.9 | 57.6 to 69.2 | 30.8 to 42.4 |
| 123.0 | 29.5 | 62.0 | 38.0 | 32.4 to 99.9 | 0.1 to 67.6 | 49.6 to 74.4 | 25.6 to 50.4 | 55.7 to 68.3 | 31.7 to 44.3 |
| 159.6 | 39.5 | 60.6 | 39.4 | 28.2 to 99.9 | 0.1 to 71.8 | 47.0 to 74.3 | 25.7 to 53.0 | 53.7 to 67.5 | 32.5 to 46.3 |
| 203.2 | 49.3 | 59.3 | 40.7 | 21.3 to 99.9 | 0.1 to 78.7 | 44.4 to 74.4 | 25.6 to 55.6 | 51.7 to 67.0 | 33.0 to 48.3 |
| 254.4 | 59.0 | 58.1 | 41.9 | 0.1 to 99.9 | 0.1 to 99.9 | 41.5 to 75.1 | 24.9 to 58.5 | 49.6 to 66.7 | 33.3 to 50.4 |
| 316.1 | 68.9 | 57.0 | 43.0 | 0.1 to 99.9 | 0.1 to 99.9 | 38.3 to 76.5 | 23.5 to 61.7 | 47.4 to 66.8 | 33.2 to 52.6 |
| 388.2 | 78.7 | 56.1 | 43.9 | 0.1 to 99.9 | 0.1 to 99.9 | 34.4 to 79.4 | 20.6 to 65.6 | 44.9 to 67.6 | 32.4 to 55.1 |

XIII. Production of 1,1-difluoropropene (HFO-1252zc)

It has been found that azeotrope or azeotrope-like compositions of 1,1-difluoropropene (HFO-1252zc) and other compounds, such as hydrogen chlorotrifluoroethylene (CTFE), hexafluoropropylene (HFP), and/or cis-1,2-difluoroethylene (HFO-1132(Z)) may be formed or otherwise encountered during production of 1,1-difluoropropene (HFO-1252zc).

Specifically, the azeotrope or azeotrope-like compositions of 1,1-difluoropropene (HFO-1252zc) and chlorotrifluoroethylene (CTFE), hexafluoropropylene (HFP), and/or cis-1,2-difluoroethylene (HFO-1132(Z)) are formed in the production of 1,1-difluoropropene (HFO-1252zc), where each of chlorotrifluoroethylene (CTFE), hexafluoropropylene (HFP), and/or cis-1,2-difluoroethylene (HFO-1132(Z)) may be deemed (i) an unreacted reactant, (ii) a desirable reaction product, (iii) a desirable intermediate, or (iv) an undesirable intermediate. In the case (i), the secondary compound may be separated from the 1,1-difluoropropene (HFO-1252zc), and subsequently recycled back into the process when forming the 1,1-difluoropropene (HFO-1252zc). In the case if (ii), the secondary compound may be separated from the 1,1-difluoropropene (HFO-1252zc) and recovered as a desirable reaction product. In the case of (iii), the secondary compound may be separated from the 1,1-difluoropropene (HFO-1252zc) and recovered as a desirable intermediate, which may undergo further reactions/processes to result in a desirable reaction product. In the case of (iv) the secondary compound may be separated from the 1,1-difluoropropene (HFO-1252zc) and recovered, in order to enhance the purity of the 1,1-difluoropropene (HFO-1252zc) product. In each of the foregoing cases, separation of the secondary compound from the 1,1-difluoropropene (HFO-1252zc) is required, as will be described with further detail herein.

### XIV. Separating Azeotrope and Azeotrope-Like Compositions Via Pressure Swing Distillation

The present disclosure also provides a method for separating an azeotrope or azeotrope-like composition of 1,1-difluoropropene (HFO-1252zc) and chlorotrifluoroethylene (CTFE), hexafluoropropylene (HFP), and/or cis-1,2-difluoroethylene (HFO-1132(Z)) via pressure swing distillation, as discussed below and in Example 12.

In a first step, a mixture of 1,1-difluoropropene (HFO-1252zc) and chlorotrifluoroethylene (CTFE), hexafluoropropylene (HFP), and/or cis-1,2-difluoroethylene (HFO-1132(Z)) is conveyed to a first, low-pressure column having a first pressure to provide a first distillate and a first bottoms product. The first bottoms product may be an enriched stream of 1,1-difluoropropene (HFO-1252zc), which is collected from the bottom of the low-pressure column. The first distillate is an azeotrope or azeotrope-like composition of 1,1-difluoropropene (HFO-1252zc) and chlorotrifluoroethylene (CTFE), hexafluoropropylene (HFP), and/or cis-1,2-difluoroethylene (HFO-1132(Z)) and is collected from the top of the low-pressure column and conveyed to a second, high-pressure column to provide a second distillate from the top of the high-pressure column and a second bottoms product from the bottom of the high-pressure column. The second bottoms product is an enriched steam of chlorotrifluoroethylene (CTFE), hexafluoropropylene (HFP), and/or cis-1,2-difluoroethylene (HFO-1132(Z)) and is collected from the high-pressure column. The second distillate includes an azeotrope or azeotrope-like composition consisting essentially of effective amounts of 1,1-difluoropropene (HFO-1252zc) and chlorotrifluoroethylene (CTFE), hexafluoropropylene (HFP), and/or cis-1,2-difluoroethylene (HFO-1132(Z)), which may be recycled back to the low-pressure column.

The above process may be modified to reverse the sequence of the low and high-pressure columns, wherein the high-pressure column is the first column and the low-pressure column is the second column and, when so modified, the first bottoms product is an enriched stream of 1,1-difluoropropene (HFO-1252zc) and the second bottoms stream is an enriched stream of chlorotrifluoroethylene (CTFE), hexafluoropropylene (HFP), and/or cis-1,2-difluoroethylene (HFO-1132(Z)).

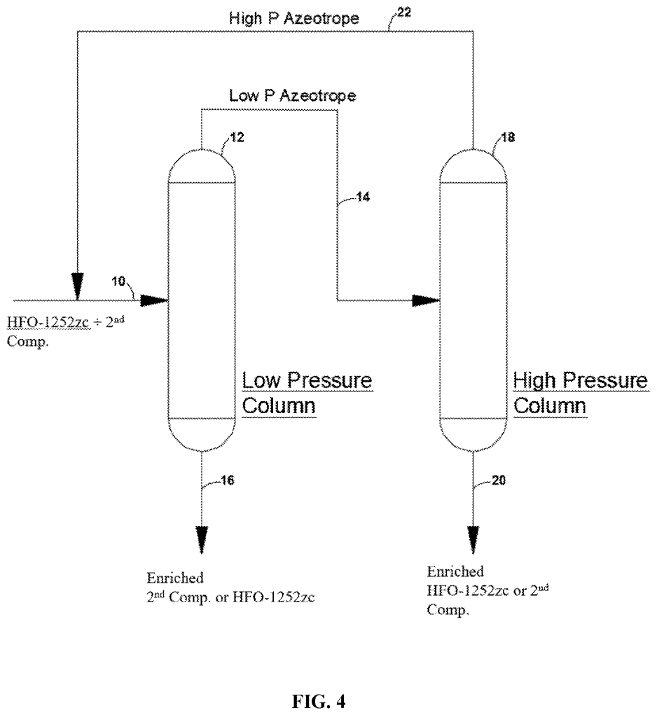

A schematic of an exemplary separation apparatus is provided in FIG. 4. Referring to this figure, a mixture of 1,1-difluoropropene (HFO-1252zc) and chlorotrifluoroethylene (CTFE), hexafluoropropylene (HFP), and/or cis-1,2-difluoroethylene (HFO-1132(Z)) is fed into a first low-pressure column 12 as feed stream 10. The low-pressure column 12 provides a first distillate stream 14 which is an azeotrope or azeotrope-like composition of 1,1-difluoropropene (HFO-1252zc) and chlorotrifluoroethylene (CTFE), hexafluoropropylene (HFP), and/or cis-1,2-difluoroethylene (HFO-1132(Z)) and a first bottoms product 16 which may be enriched in, or consist essentially of, 1,1-difluoropropene (HFO-1252zc). The first distillate stream 14 is then conveyed to a high-pressure column 18 to provide a second distillate stream 22 which is an azeotrope or azeotrope-like composition of 1,1-difluoropropene (HFO-1252zc) and chlorotrifluoroethylene (CTFE), hexafluoropropylene (HFP), and/or cis-1,2-difluoroethylene (HFO-1132(Z)), and a second bottoms product 20. The second bottoms product 20 is enriched in, or consists essentially of, chlorotrifluoroethylene (CTFE), hexafluoropropylene (HFP), and/or cis-1,2-difluoroethylene (HFO-1132(Z)). The second distillate stream 22 may optionally be fed back into feed stream 10 and thereby recycled into the low-pressure column 12.

### XV. Separating Azeotrope and Azeotrope-Like Compositions Via Extractive Distillation

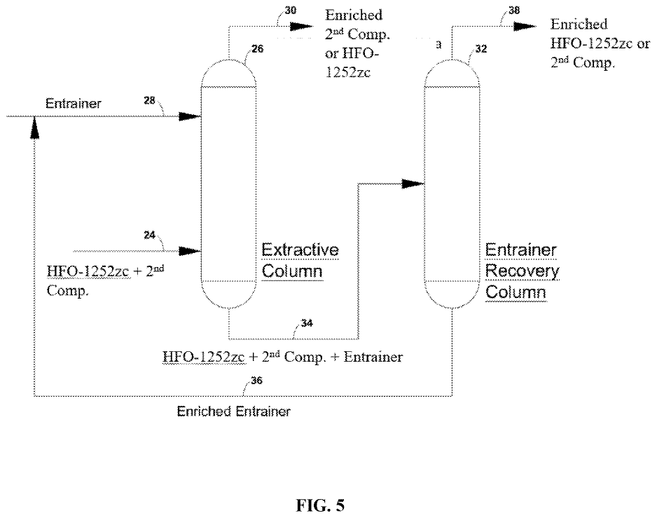

The present disclosure provides a method for separating an azeotrope or azeotrope-like composition of 1,1-difluoropropene (HFO-1252zc)) and chlorotrifluoroethylene (CTFE), hexafluoropropylene (HFP), and/or cis-1,2-difluoroethylene (HFO-1132(Z)) via extractive distillation. Example 5 discusses one such embodiment. A schematic of an exemplary separation apparatus is provided in FIG. 5.

For instance, the extractive distillation method may include conveying a product mixture and an entrainer fluid to a first column. The extractive distillation method may further include collecting a first distillate from the first column comprising a first component of the azeotrope or azeotrope-like composition. The extractive distillation method may further include collecting a first bottoms product from the first column comprising a mixture of the entrainer and a second component of the azeotrope or azeotrope-like composition. The extractive distillation method may further include conveying the first bottoms product to a second column to separate the entrainer and the second component of the azeotrope or azeotrope-like composition. The extractive distillation method may further include removing a composition consisting essentially of the second component of the azeotrope or azeotrope-like composition as a second distillate from the second column.

A schematic of an exemplary separation apparatus is provided in FIG. 5. Referring to this figure, product stream 24, which includes at least the azeotrope or azeotrope-like composition of 1,1-difluoropropene (HFO-1252zc)) and chlorotrifluoroethylene (CTFE), hexafluoropropylene (HFP), and/or cis-1,2-difluoroethylene (HFO-1132(Z)) is conveyed to extractive column 26 along with an entrainer fluid 28. The entrainer fluid 28 is a composition where one of the components of the azeotrope or azeotrope-like composition of 1,1-difluoropropene (HFO-1252zc)) and chlorotrifluoroethylene (CTFE), hexafluoropropylene (HFP), and/or cis-1,2-difluoroethylene (HFO-1132(Z)) has a higher affinity for the entrainer fluid, as compared to the other. Here, one of the components of the azeotrope or azeotrope-like composition dissolves in the entrainer fluid 28 more readily than the other component. The entrainer fluid 28 therefore may be used to “break” the azeotrope or azeotrope-like composition into individual components, based upon the affinity/solubility difference, and therefore, utilized to selectively separate each of the components of the azeotrope or azeotrope-like composition. For example, the entrainer fluid 28 may have a higher affinity for 1,1-difluoropropene (HFO-1252zc) and may act as a selective solute for the 1,1-difluoropropene (HFO-1252zc) of the azeotrope or azeotrope-like composition. In another example, the entrainer fluid 28 may have a higher affinity for the chlorotrifluoroethylene (CTFE), hexafluoropropylene (HFP), and/or cis-1,2-difluoroethylene (HFO-1132(Z)) and may act as a selective solute for the chlorotrifluoroethylene (CTFE), hexafluoropropylene (HFP), and/or cis-1,2-difluoroethylene (HFO-1132(Z)) of the azeotrope or azeotrope-like composition.

Extractive column 26 is operated such that the operational parameters (temperature and pressure) separate the mixture of the entrainer fluid 28 and the first/dissolved component of the azeotrope or azeotrope-like composition from the second component of the azeotrope or azeotrope-like composition. For example, in the case where the entrainer fluid 28 has a higher affinity for 1,1-difluoropropene (HFO-1252zc)), 1,1-difluoropropene (HFO-1252zc) and the entrainer fluid 28 are recovered in bottoms product 34 from extractive column 26, while enriched 1chlorotrifluoroethylene (CTFE), hexafluoropropylene (HFP), and/or cis-1,2-difluoroethylene (HFO-1132(Z)) is recovered as the distillate 30. In another example, in the case where the entrainer fluid 28 has a higher affinity for chlorotrifluoroethylene (CTFE), hexafluoropropylene (HFP), and/or cis-1,2-difluoroethylene (HFO-1132(Z)), chlorotrifluoroethylene (CTFE), hexafluoropropylene (HFP), and/or cis-1,2-difluoroethylene (HFO-1132(Z)) and the entrainer fluid 28 are recovered in bottoms product 34, while enriched 1,1-difluoropropene (HFO-1252zc)) is recovered as the distillate 30.

In a second step, the bottoms product 34 containing each of the mixture of the entrainer fluid and the dissolved first component of the azeotrope or azeotrope-like composition is conveyed to recovery column 32. Recovery column 32 is operated such that the operational parameters (temperature and pressure) separate the entrainer fluid and the dissolved component of the azeotrope or azeotrope-like composition from one another, where the enriched entrainer fluid is recovered as bottoms product 34 from extractive column 26, and the enriched either 1,1-difluoropropene (HFO-1252zc)) or chlorotrifluoroethylene (CTFE), hexafluoropropylene (HFP), and/or cis-1,2-difluoroethylene (HFO-1132(Z)) is recovered as in the distillate 38. The recovered enriched entrainer fluid may be recycled and used as entrainer fluid 28 in the extractive column 26, as described previously.

### EXAMPLES

### Example 1: Measurement and Characterization of Azeotrope and Azeotrope-Like Compositions of 1,1-difluoropropene (HFO-1252zc) and chlorotrifluoroethylene (CTFE)

A set of volume calibrated PTx cells were used to measure azeotrope and azeotrope-like compositions of 1,1-difluoropropene (HFO-1252zc) and chlorotrifluoroethylene (CTFE). Mixtures of HFO-1252zc and CTFE were gravimetrically prepared into evacuated PTx cells; two cells were reserved for measuring each pure component. Once prepared, each of up to eight cells of differing compositions were inserted into a thermostatted chamber. In the chamber, each cell was attached to an instrumentation manifold equipped with calibrated pressure transducers and resistance temperature detectors (RTD); this provided a means to measure and record the total saturation pressure of each cell's contents at its local temperature.

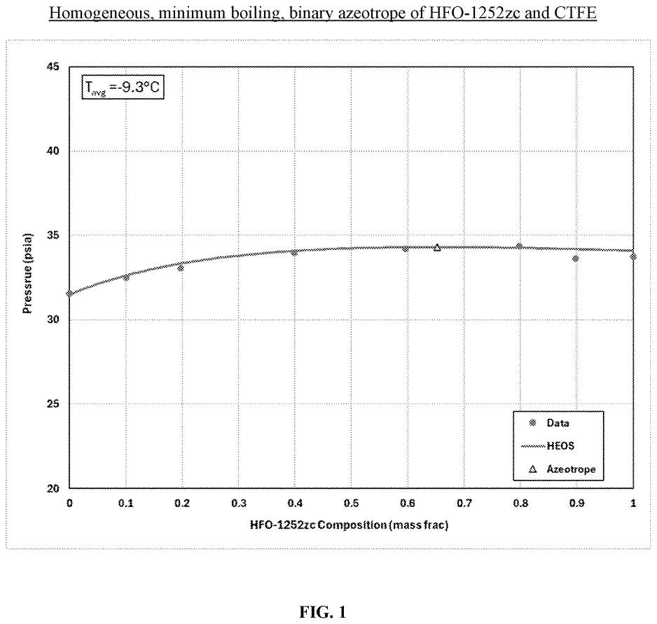

To establish equilibrium at a target temperature, the set point of the chamber was adjusted, yielding an average temperature (T_{avg}) of −9.3° C. across all of the PTx cells. Once at equilibrium, recognized as when temperature and pressures of each cell remain stable for several hours, the local temperature and saturation pressures of each cell were recorded. From these pressure-temperature-composition data, the binary interaction parameters of HFO-1252zc and CTFE for the Helmholtz Energy Equation of State (HEOS) were identified. As indicated by the maximum pressure shown in FIG. 1 a homogeneous, minimum boiling azeotrope composition of about 65.3 wt. % HFO-1252zc and about 34.7 wt. % CTFE was formed based on the data presented in Table 4

TABLE 4
PTx Study of HFO-1252zc and CTFE at an average temperature
of −9.3° C. Maximum pressure observed between
59.6 wt % HFO-1252zc and 89.9 wt % HFO-1252zc, azeotrope
formed at about 65.3 wt % HFO-1252zc.

| HFO-1252zc Composition | CTFE Composition | Pressure |
|---|---|---|
| (wt %) | (wt %) | (psia) |
| 100.0 | 0.0 | 33.7 |
| 89.9 | 10.2 | 33.6 |
| 79.8 | 20.2 | 34.4 |
| 59.6 | 40.4 | 34.2 |
| 39.8 | 60.2 | 34.0 |
| 19.7 | 80.3 | 33.1 |
| 10.0 | 90.0 | 32.5 |
| 0.0 | 100.0 | 31.6 |

### Example 2: Azeotrope Locus

The procedure of Example 1 was repeated for each of the temperatures indicated in Table 5 below to generate the azeotropic and azeotrope-like compositional ranges.

TABLE 5
Azeotrope Locus

| | | Azeotropic | | Azeotropic-Like Composition Range | | |
|---|---|---|---|---|---|---|
| Saturation | Saturation | Composition | | Glide <0.5° C. | Rel. Vol. <1.1 | Rel. Vol. <1.05 |
| Pressure | Temperature | Wt. % | Wt. % | Wt. % | Wt. % | Wt. % |
| (psia) | (° C.) | HFO-1252zc | CTFE | HFO-1252zc | HFO-1252zc | HFO-1252zc |
| 15.6 | −29.4 | 72.9 | 27.1 | 0.1 to 99.9 | 36.0 to 99.9 | 50.4 to 99.9 |
| 23.8 | −19.1 | 68.5 | 31.5 | 0.1 to 99.9 | 32.2 to 99.9 | 46.4 to 99.9 |
| 34.4 | −9.3 | 65.3 | 34.7 | 0.1 to 99.9 | 28.9 to 99.9 | 43.1 to 99.9 |
| 47.9 | 0.3 | 62.8 | 37.2 | 0.1 to 99.9 | 25.9 to 99.9 | 40.4 to 99.9 |
| 65.2 | 9.9 | 60.9 | 39.1 | 0.1 to 99.9 | 23.2 to 99.9 | 37.9 to 99.9 |
| 87.2 | 19.7 | 59.5 | 40.5 | 0.1 to 99.9 | 20.4 to 99.9 | 35.6 to 99.9 |
| 114.8 | 29.5 | 58.4 | 41.6 | 0.1 to 99.9 | 17.7 to 99.9 | 33.3 to 99.9 |
| 148.6 | 39.5 | 57.7 | 42.3 | 0.1 to 99.9 | 14.8 to 99.9 | 31.0 to 99.9 |
| 188.7 | 49.3 | 57.3 | 42.7 | 0.1 to 99.9 | 11.8 to 99.9 | 28.8 to 99.9 |
| 235.9 | 59.0 | 57.1 | 42.9 | 0.1 to 99.9 | 8.4 to 99.9 | 36.3 to 99.9 |
| 292.3 | 68.9 | 57.2 | 42.8 | 0.1 to 99.9 | 4.2 to 99.9 | 23.3 to 99.9 |
| 358.8 | 78.8 | 57.5 | 42.5 | 0.1 to 99.9 | 0.1 to 99.9 | 19.2 to 99.9 |

In view of the above data, temperature glide and relative volatility were applied to determine the azeotrope and azeotrope-like compositions.

The temperature glide and relative volatility of a mixture may be derived from thermodynamic measurements, such as those collected via PTx, subject to material balance and thermodynamic constraints. Several methods for deriving temperature glide from thermodynamic measurements are described in Sandler, S. I. (2006). Chapter 10: Vapor-Liquid Equilibrium in Mixtures. In Chemical, Biochemical, and Engineering Thermodynamics (4th ed., pp. 489-574) which includes constraining thermodynamic consistency through the fundamental Gibbs-Duhem relationship and resolving the vapor phase composition, from the measurements, through combined mass balance and equilibrium criteria (frequently referred to as the Rachford-Rice equation or algorithm). Through this derivation, the relationship between equilibrium compositions, temperatures, and pressures are established, permitting temperature glide and relative volatility to be evaluated.

For a given composition, the temperature glide, by definition, is the difference between the saturated vapor temperature and the saturated liquid temperature at a fixed pressure in thermodynamic equilibrium. Consequently, an azeotrope composition has a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This is the broad azeotrope-like range.

The relative volatility, by definition, is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This is the intermediate azeotrope-like range.

Additionally, it has been identified that a relative volatility of 1.05 is substantially close to 1.0 and therefore compositions that satisfy such relative volatility are considered azeotrope-like. This is the narrow azeotrope-like range.

### Example 3: Measurement and Characterization of Azeotrope and Azeotrope-Like Compositions of hexafluoropropylene (HFP) and 1,1-difluoropropene (HFO-1252zc)

A set of volume calibrated PTx cells were used to measure azeotrope and azeotrope-like compositions of hexafluoropropylene (HFP) and 1,1-difluoropropene (HFO-1252zc). Mixtures of HFP and HFO-1252zc were gravimetrically prepared into evacuated PTx cells; two cells were reserved for measuring each pure component. Once prepared, each of up to eight cells of differing compositions were inserted into a thermostatted chamber. In the chamber, each cell was attached to an instrumentation manifold equipped with calibrated pressure transducers and resistance temperature detectors (RTD); this provided a means to measure and record the total saturation pressure of each cell's contents at its local temperature.

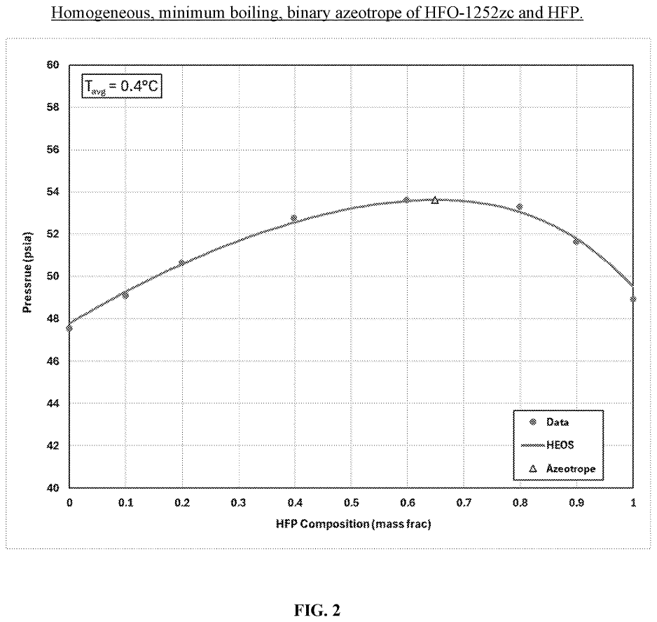

To establish equilibrium at a target temperature, the set point of the chamber was adjusted, yielding an average temperature (T_{avg}) of 0.4° C. across all of the PTx cells. Once at equilibrium, recognized as when temperature and pressures of each cell remain stable for several hours, the local temperature and saturation pressures of each cell were recorded. From these pressure-temperature-composition data, the binary interaction parameters of HFP and HFO-1252zc for the Helmholtz Energy Equation of State (HEOS) were identified. As indicated by the maximum pressure shown in FIG. 2, a homogeneous, minimum boiling azeotrope composition of about 64.9 wt. % HFP and about 35.1 wt. % HFO-1252zc was formed based on the data presented in Table 6

TABLE 6
PTx Study of HFP and HFO-1252zc at an average temperature of
0.4° C. Maximum pressure observed between 39.8 wt % HFP
and 79.8 wt % HFP, azeotrope formed at about 64.9 wt % HFP.

| HFP Composition | HFO-1252zc Composition | Pressure |
|---|---|---|
| (wt %) | (wt %) | (psia) |
| 100.0 | 0.0 | 48.9 |
| 89.9 | 10.1 | 51.7 |
| 79.8 | 20.2 | 53.3 |
| 59.8 | 40.2 | 53.6 |
| 39.8 | 60.2 | 52.8 |
| 20.0 | 80.0 | 50.6 |
| 10.0 | 90.0 | 49.1 |
| 0.0 | 100.0 | 47.6 |

### Example 4: Azeotrope Locus

The procedure of Example 3 was repeated for each of the temperatures indicated in Table 7 below to generate the azeotropic and azeotrope-like compositional ranges.

TABLE 7
Azeotrope Locus

| | | Azeotropic | | | | |
|---|---|---|---|---|---|---|
| Saturation | Saturation | Composition | | Azeotropic-Like Composition Range | | |
| Pressure | Temperature | Wt. % | Wt. % | Glide <0.5° C. | Rel. Vol. <1.1 | Rel. Vol. <1.05 |
| (psia) | (° C.) | HFP | HFO-1252zc | Wt. % HFP | Wt. % HFP | Wt. % HFP |
| 17.4 | −29.3 | 59.7 | 40.3 | 0.1 to 99.9 | 49.8 to 69.5 | 54.7 to 64.7 |
| 26.4 | −19.2 | 61.6 | 38.4 | 0.1 to 99.9 | 51.0 to 72.2 | 56.3 to 67.0 |
| 38.2 | −9.3 | 63.4 | 36.6 | 0.1 to 99.9 | 51.9 to 74.8 | 57.6 to 69.1 |
| 53.6 | 0.4 | 64.9 | 35.1 | 0.1 to 99.9 | 52.6 to 77.4 | 58.6 to 71.2 |
| 73.2 | 10.1 | 66.4 | 33.6 | 0.1 to 99.9 | 53.1 to 80.0 | 59.6 to 73.2 |
| 96.9 | 19.5 | 67.7 | 32.3 | 0.1 to 99.9 | 53.3 to 82.7 | 60.4 to 75.1 |
| 127.6 | 29.4 | 69.0 | 31.0 | 0.1 to 99.9 | 53.3 to 85.9 | 61.0 to 77.3 |
| 165.4 | 39.4 | 70.3 | 29.7 | 0.1 to 99.9 | 52.9 to 89.9 | 61.4 to 79.6 |
| 210.7 | 49.2 | 71.6 | 28.4 | 0.1 to 99.9 | 52.1 to 95.4 | 61.6 to 82.3 |
| 263.7 | 58.9 | 72.9 | 27.1 | 0.1 to 99.9 | 50.7 to 99.9 | 61.5 to 85.6 |
| 327.9 | 68.8 | 74.3 | 25.7 | 0.1 to 99.9 | 48.0 to 99.9 | 60.7 to 91.0 |
| 404.1 | 78.7 | 76.1 | 23.9 | 0.1 to 99.9 | 42.5 to 99.9 | 58.3 to 99.9 |

In view of the above data, temperature glide and relative volatility were applied to determine the azeotrope and azeotrope-like compositions.

The temperature glide and relative volatility of a mixture may be derived from thermodynamic measurements, such as those collected via PTx, subject to material balance and thermodynamic constraints. Several methods for deriving temperature glide from thermodynamic measurements are described in Sandler, S. I. (2006). Chapter 10: Vapor-Liquid Equilibrium in Mixtures. In Chemical, Biochemical, and Engineering Thermodynamics (4th ed., pp. 489-574) which includes constraining thermodynamic consistency through the fundamental Gibbs-Duhem relationship and resolving the vapor phase composition, from the measurements, through combined mass balance and equilibrium criteria (frequently referred to as the Rachford-Rice equation or algorithm). Through this derivation, the relationship between equilibrium compositions, temperatures, and pressures are established, permitting temperature glide and relative volatility to be evaluated.

For a given composition, the temperature glide, by definition, is the difference between the saturated vapor temperature and the saturated liquid temperature at a fixed pressure in thermodynamic equilibrium. Consequently, an azeotrope composition has a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This is the broad azeotrope-like range.

The relative volatility, by definition, is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This is the intermediate azeotrope-like range.

Additionally, it has been identified that a relative volatility of 1.05 is substantially close to 1.0 and therefore compositions that satisfy such relative volatility are considered azeotrope-like. This is the narrow azeotrope-like range.

### Example 5: Measurement and Characterization of Azeotrope and Azeotrope-Like Compositions of 1,1-difluoropropene (HFO-1252zc) and cis-1,2-difluoroethylene (HFO-1132(Z))

A set of volume calibrated PTx cells were used to measure azeotrope and azeotrope-like compositions of 1,1-difluoropropene (HFO-1252zc) and cis-1,2-difluoroethylene (HFO-1132(Z)). Mixtures of HFO-1252zc and HFO-1132(Z) were gravimetrically prepared into evacuated PTx cells; two cells were reserved for measuring each pure component. Once prepared, each of up to eight cells of differing compositions were inserted into a thermostatted chamber. In the chamber, each cell was attached to an instrumentation manifold equipped with calibrated pressure transducers and resistance temperature detectors (RTD); this provided a means to measure and record the total saturation pressure of each cell's contents at its local temperature.

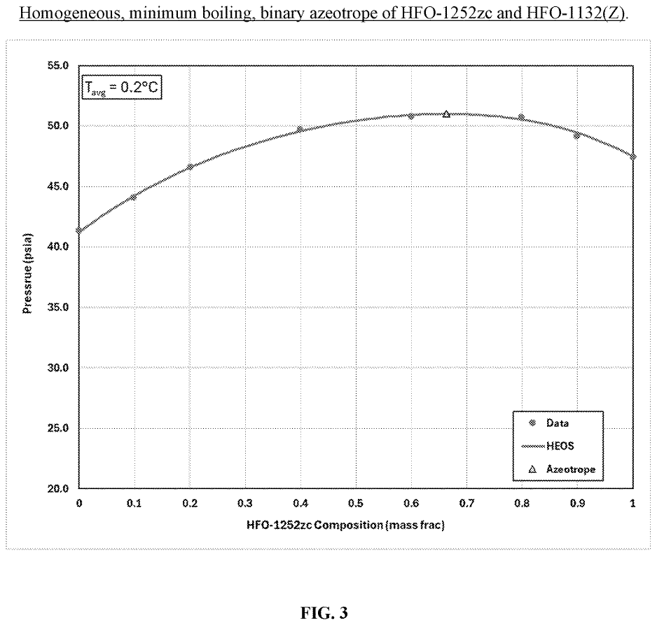

To establish equilibrium at a target temperature, the set point of the chamber was adjusted, yielding an average temperature (T_{avg}) of 0.2° C. across all of the PTx cells. Once at equilibrium, recognized as when temperature and pressures of each cell remain stable for several hours, the local temperature and saturation pressures of each cell were recorded. From these pressure-temperature-composition data, the binary interaction parameters of HFO-1252zc and HFO-1132(Z) for the Helmholtz Energy Equation of State (HEOS) were identified. As indicated by the maximum pressure shown in FIG. 3, a homogeneous, minimum boiling azeotrope composition of about 66.3 wt. % HFO-1252zc and about 33.7 wt. % HFO-1132(Z) was formed based on the data presented in Table 8.

TABLE 8
PTx Study of HFO-1252zc and HFO-1132(Z) at an average
temperature of 0.2° C. Maximum pressure observed
between 39.8 wt % HFO-1252zc and 79.9 wt % HFO-1252zc,
azeotrope formed at about 66.3 wt % HFO-1252zc.

| HFO-1252zc Composition | HFO-1132(Z) Composition | Pressure |
|---|---|---|
| (wt %) | (wt %) | (psia) |
| 100.0 | 0.0 | 47.5 |
| 89.9 | 10.1 | 49.2 |
| 79.9 | 20.1 | 50.8 |
| 60.0 | 40.0 | 50.8 |
| 39.8 | 60.2 | 49.7 |
| 20.1 | 79.9 | 46.6 |
| 9.9 | 90.1 | 44.1 |
| 0.0 | 100.0 | 41.3 |

### Example 6: Azeotrope Locus

The procedure of Example 5 was repeated for each of the temperatures indicated in Table 9 below to generate the azeotropic and azeotrope-like compositional ranges.

TABLE 9
Azeotrope Locus

| | | | | Azeotropic-Like Composition Range | | |
|---|---|---|---|---|---|---|
| Saturation | Saturation | Azeotropic Composition | | Glide <0.5° C. | Rel. Vol. <1.1 | Rel. Vol. <1.05 |
| Pressure | Temperature | Wt. % | Wt. % | Wt. % | Wt. % | Wt. % |
| (psia) | (° C.) | HFO-1252zc | HFO-1132(Z) | HFO-1252zc | HFO-1252zc | HFO-1252zc |
| 16.7 | −29.2 | 71.0 | 29.0 | 49.6 to 99.9 | 63.3 to 78.7 | 67.1 to 74.9 |
| 25.2 | −19.2 | 69.4 | 30.6 | 47.1 to 99.9 | 61.1 to 77.7 | 65.2 to 73.6 |
| 36.5 | −9.4 | 67.8 | 32.2 | 44.5 to 99.9 | 58.9 to 76.8 | 63.3 to 72.4 |
| 51.0 | 0.2 | 66.3 | 33.7 | 41.8 to 99.9 | 56.7 to 76.0 | 61.4 to 71.3 |
| 69.7 | 9.9 | 64.9 | 35.1 | 39.0 to 99.9 | 54.4 to 75.3 | 59.5 to 70.2 |
| 93.3 | 19.6 | 63.4 | 36.6 | 35.9 to 99.9 | 52.1 to 74.8 | 57.6 to 69.2 |
| 123.0 | 29.5 | 62.0 | 38.0 | 32.4 to 99.9 | 49.6 to 74.4 | 55.7 to 68.3 |
| 159.6 | 39.5 | 60.6 | 39.4 | 28.2 to 99.9 | 47.0 to 74.3 | 53.7 to 67.5 |
| 203.2 | 49.3 | 59.3 | 40.7 | 21.3 to 99.9 | 44.4 to 74.4 | 51.7 to 67.0 |
| 254.4 | 59.0 | 58.1 | 41.9 | 0.1 to 99.9 | 41.5 to 75.1 | 49.6 to 66.7 |
| 316.1 | 68.9 | 57.0 | 43.0 | 0.1 to 99.9 | 38.3 to 76.5 | 47.4 to 66.8 |
| 388.2 | 78.7 | 56.1 | 43.9 | 0.1 to 99.9 | 34.4 to 79.4 | 44.9 to 67.6 |

In view of the above data, temperature glide and relative volatility were applied to determine the azeotrope and azeotrope-like compositions.

The temperature glide and relative volatility of a mixture may be derived from thermodynamic measurements, such as those collected via PTx, subject to material balance and thermodynamic constraints. Several methods for deriving temperature glide from thermodynamic measurements are described in Sandler, S. I. (2006). Chapter 10: Vapor-Liquid Equilibrium in Mixtures. In Chemical, Biochemical, and Engineering Thermodynamics (4th ed., pp. 489-574) which includes constraining thermodynamic consistency through the fundamental Gibbs-Duhem relationship and resolving the vapor phase composition, from the measurements, through combined mass balance and equilibrium criteria (frequently referred to as the Rachford-Rice equation or algorithm). Through this derivation, the relationship between equilibrium compositions, temperatures, and pressures are established, permitting temperature glide and relative volatility to be evaluated.

For a given composition, the temperature glide, by definition, is the difference between the saturated vapor temperature and the saturated liquid temperature at a fixed pressure in thermodynamic equilibrium. Consequently, an azeotrope composition has a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This is the broad azeotrope-like range.

The relative volatility, by definition, is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This is the intermediate azeotrope-like range.

Additionally, it has been identified that a relative volatility of 1.05 is substantially close to 1.0 and therefore compositions that satisfy such relative volatility are considered azeotrope-like. This is the narrow azeotrope-like range.

### Example 7: Pressure Swing Separation

A well-known consequence of azeotropic mixtures is the inability to fully separate its constituents in a single continuous distillation operation. For example, separation of a 50/50 mass % mixture of 1,1-difluoropropene (HFO-1252zc) and another compound, such as chlorotrifluoroethylene (CTFE), hexafluoropropylene (HFP), and/or cis-1,2-difluoroethylene (HFO-1132(Z)) by a continuous distillation column held at 14.7 psia, exhibiting azeotropic behavior as described by Examples 1-6, would be bounded by compositions between the pure second component (i.e., 0 mass % chlorotrifluoroethylene (CTFE), hexafluoropropylene (HFP), and/or cis-1,2-difluoroethylene (HFO-1132(Z)) endpoint and the minimum boiling azeotropic composition. In other words, distillation of a mixture at these conditions would be unable to produce 1,1-difluoropropene (HFO-1252zc) in a purity greater than the maximum percent of such Azeotrope/azeotrope like composition. To address this fundamental barrier of azeotropes and attain both purer 1,1-difluoropropene (HFO-1252zc) and the secondary components chlorotrifluoroethylene (CTFE), hexafluoropropylene (HFP), and/or cis-1,2-difluoroethylene (HFO-1132(Z)), a different separation strategy must be realized.

The azeotropic composition of 1,1-difluoropropene (HFO-1252zc) and another compound, such as chlorotrifluoroethylene (CTFE), hexafluoropropylene (HFP), and/or cis-1,2-difluoroethylene (HFO-1132(Z)) is sensitive to pressure. This sensitivity can be exploited to support better separation through pressure swing distillation. In this system, a pressure-sensitive azeotrope is separated using two distillation columns in sequence, one at an arbitrary, relatively lower pressure and one at an arbitrary, relatively higher pressure. The columns may be disposed such that the lower pressure column is first in the sequence. Alternatively, the higher-pressure column may be first in the sequence. For the purposes of this example, with reference to FIG. 4, the columns are disposed with the lower pressure column first in the sequence.

A mixture of 1,1-difluoropropene (HFO-1252zc) and another component, such as chlorotrifluoroethylene (CTFE), hexafluoropropylene (HFP), and/or cis-1,2-difluoroethylene (HFO-1132(Z)) is first subjected to distillation at a lower pressure. The particular composition of the mixture may be tailored as needed.

The feed composition of stream 10 has not yet reached the azeotrope point for the column pressure. As such, the mixture may be separated into fractions enriched in one component of the mixture and the azeotrope or azeotrope-like composition. Here, fractions enriched in the lower boiling point component, component A, are collected as the bottoms shown as stream 16 in FIG. 4. The azeotrope or azeotrope-like composition is the distillate from the low-pressure column 12 shown in FIG. 4. This mixture is then passed to a column 18 at an arbitrary higher pressure, following stream 14 in FIG. 4.

When composition of stream 14 is brought to the higher pressure of column 18, its composition relative to the higher-pressure azeotropic composition is now lower. This permits fractions enriched in the other component of the mixture to be collected. In this example, fractions enriched in Component B are collected as the bottoms product shown as stream 20 in FIG. 4. As with the lower pressure column, the distillate comprises the azeotrope or azeotrope-like mixture. This mixture may be recycled back to co-mingle with low pressure column feed following stream 22 in FIG. 4.

In this way, the barrier of the azeotrope is addressed, using the sensitivity of its composition to the column conditions, to produce two streams each enriched in one of the components. It is important to note that the details in this example are meant to be illustrative. Depending on the context of the mixture, the column conditions and configurations can be designed to support nearly any desired purities of 1,1-difluoropropene (HFO-1252zc) and another component, such as chlorotrifluoroethylene (CTFE), hexafluoropropylene (HFP), cis-1,2,3,3,3-pentafluoro propene (HFO-1225ye (Z)), 1-fluoropropene (HFO-1261ze), HF, water (H₂O), chloroethyne (C2HCl), trans-1,2-difluoropropene (HFO-1252ye (E)), 1-chloro-1-fluoroethylene (HCFO-1131a), 3,3,3-trifluoropropene (HFO-1243zf), and/or cis-1,2-difluoroethylene (HFO-1132(Z)).

### Example 8: Extractive Distillation

An Azeotrope or azeotrope-like composition of 11,1-difluoropropene (HFO-1252zc) and another component, such as chlorotrifluoroethylene (CTFE), hexafluoropropylene (HFP), and/or cis-1,2-difluoroethylene (HFO-1132(Z)) is separated by extractive distillation. Initially, a stream containing an azeotrope or azeotrope-like composition of 1,1-difluoropropene (HFO-1252zc) and another component, such as chlorotrifluoroethylene (CTFE), hexafluoropropylene (HFP), and/or cis-1,2-difluoroethylene (HFO-1132(Z)) is fed to a first distillation column, along with an entrainer fluid. The extraction column is operated at a suitable temperature and pressure. The first distillation column acts as an extraction column, where the entrainer fluid and one of the components of the azeotrope or azeotrope-like composition (e.g., component A) is recovered in the bottoms product from the extraction column, and the other component of the azeotrope or azeotrope-like composition (e.g., component B). Here, the entrainer fluid is selected based upon various thermodynamic properties which includes an affinity difference between component A to dissolve in the entrainer fluid vs. component B, where substantially all of the component A is dissolved in the entrainer fluid, while little to none of the component B is dissolved in the entrainer fluid. The column is operated based upon thermodynamic differences between entrainer/component A and the component B, similar to those described in relation to Example 3: pressure swing distillation. The extraction column is operated such that distillate comprises substantially all of the component B, and the bottoms product comprises substantially all of the entrainer fluid and dissolved component A.

The recovered entrainer fluid and dissolved component A is fed to a second recovery column operated at a suitable temperature and pressure. The recovery column separates the entrainer fluid from the component A, as based upon thermodynamic differences, similar to those described in relation to Example 7: pressure swing distillation. Here, substantially all of the entrainer fluid is recovered in the bottoms product from the recovery column, and substantially all of the component A is recovered in the distillate. The recovered entrainer fluid is thereafter recycled to the extraction column.

### ASPECTS

Aspect 1 is an azeotrope or azeotrope-like composition consisting essentially of effective amounts of 1,1-difluoropropene (HFO-1252zc) and one of chlorotrifluoroethylene (CTFE), hexafluoropropylene (HFP), or cis-1,2-difluoroethylene (HFO-1132(Z)).

### HFO-1252zc and CTFE

Aspect 2 is the azeotrope or azeotrope-like composition of Aspect 1, consisting essentially of 1,1-difluoropropene (HFO-1252zc) and chlorotrifluoroethylene (CTFE).

Aspect 3 is the azeotrope or azeotrope-like composition of Aspect 2, consisting essentially of from about 0.1 wt. % to about 99.9 wt. % of 1,1-difluoropropene (HFO-1252zc) and from about 0.1 wt. % to about 99.9 wt. % of chlorotrifluoroethylene (CTFE).

Aspect 4 is the azeotrope or azeotrope-like composition of Aspect 2, consisting essentially of from about 19.2 wt. % to about 99.9 wt. % of 1,1-difluoropropene (HFO-1252zc) and from about 0.1 wt. % to about 80.8 wt. % of chlorotrifluoroethylene (CTFE).

Aspect 5 is the azeotrope or azeotrope-like composition of any one of Aspects 2 through 4, consisting essentially of about 57.5 wt. % of 1,1-difluoropropene (HFO-1252zc) and about 42.5 wt. % of chlorotrifluoroethylene (CTFE) at a pressure of about 358.8 psia.

Aspect 6 is the azeotrope or azeotrope-like composition of any one of Aspects 2 through 5, wherein the azeotrope or azeotrope-like composition has a boiling point of from about −29.4° C. to about 78.8° C., at a pressure of about 15.6 psia to about 358.8 psia.

Aspect 7 is the azeotrope or azeotrope-like composition of Aspect 1, consisting essentially of 1,1-difluoropropene (HFO-1252zc) and hexafluoropropylene (HFP).

Aspect 8 is the azeotrope or azeotrope-like composition of Aspect 7, consisting essentially of from about 0.1 wt. % to about 99.9 wt. % of 1,1-difluoropropene (HFO-1252zc) and from about 0.1 wt. % to about 99.9 wt. % of hexafluoropropylene (HFP).

Aspect 9 is the azeotrope or azeotrope-like composition of Aspect 7, consisting essentially of from about 0.1 wt. % to about 57.5 wt. % of 1,1-difluoropropene (HFO-1252zc) and from about 42.5 wt. % to about 99.9 wt. % of hexafluoropropylene (HFP).

Aspect 10 is the azeotrope or azeotrope-like composition of Aspect 7, consisting essentially of from about 0.1 wt. % to about 41.7 wt. % of 1,1-difluoropropene (HFO-1252zc) and from about 58.3 wt. % to about 99.9 wt. % of hexafluoropropylene (HFP).

Aspect 11 is the azeotrope or azeotrope-like composition of any one of Aspects 7 through 10, consisting essentially of about 23.9 wt. % of 1,1-difluoropropene (HFO-1252zc) and about 76.1 wt. % of hexafluoropropylene (HFP) at a pressure of about 404.1 psia.

Aspect 12 is the azeotrope or azeotrope-like composition of any one of Aspects 7 through 11, wherein the azeotrope or azeotrope-like composition has a boiling point of from about −29.3° C. to about 78.7° C., at a pressure of about 17.4 psia to about 404.1 psia.

Aspect 13 is the azeotrope or azeotrope-like composition of Aspect 1, consisting essentially of 1,1-difluoropropene (HFO-1252zc) and cis-1,2-difluoroethylene (HFO-1132(Z)).

Aspect 14 is the azeotrope or azeotrope-like composition of Aspect 13, consisting essentially of from about 0.1 wt. % to about 99.9 wt. % of 1,1-difluoropropene (HFO-1252zc) and from about 0.1 wt. % to about 99.9 wt. % of cis-1,2-difluoroethylene (HFO-1132(Z)).

Aspect 15 is the azeotrope or azeotrope-like composition of Aspect 13, consisting essentially of from about 34.4 wt. % to about 79.4 wt. % of 1,1-difluoropropene (HFO-1252zc) and from about 20.6 wt. % to about 65.6 wt. % of cis-1,2-difluoroethylene (HFO-1132(Z)).

Aspect 16 is the azeotrope or azeotrope-like composition of Aspect 13, consisting essentially of from about 44.9 wt. % to about 67.6 wt. % of 1,1-difluoropropene (HFO-1252zc) and from about 32.4 wt. % to about 55.1 wt. % of cis-1,2-difluoroethylene (HFO-1132(Z)).

Aspect 17 is the azeotrope or azeotrope-like composition of any one of Aspects 13 through 16, consisting essentially of about 56.1 wt. % of 1,1-difluoropropene (HFO-1252zc) and about 43.9 wt. % of cis-1,2-difluoroethylene (HFO-1132(Z)) at a pressure of about 388.2 psia.

Aspect 18 is the azeotrope or azeotrope-like composition of any one of Aspects 13 through 17, wherein the azeotrope or azeotrope-like composition has a boiling point of from about −29.2° C. to about 78.7° C., at a pressure of about 16.7 psia to about 388.2 psia.

Aspect 19 is a method for producing 1,1-difluoropropene (HFO-1252zc) comprising: producing a product mixture comprising an azeotrope or azeotrope-like composition consisting essentially of effective amounts of 1,1-difluoropropene (HFO-1252zc) and any one of chlorotrifluoroethylene (CTFE), hexafluoropropylene (HFP), and/or cis-1,2-difluoroethylene (HFO-1132(Z)); and separating the 1,1-difluoropropene (HFO-1252zc) and the chlorotrifluoroethylene (CTFE), hexafluoropropylene (HFP), and/or cis-1,2-difluoroethylene (HFO-1132(Z)) to provide a product composition comprising the 1,1-difluoropropene (HFO-1252zc).

Aspect 20 is the method of Aspect 19, wherein the separating step comprises: conveying the product mixture to a first column having a first pressure; collecting a first bottoms product from the first column; conveying a first distillate from the first column to a second column having a second pressure to provide a second distillate and a second bottoms product, the second distillate comprising the azeotrope or azeotrope-like composition; and collecting a second bottoms product from the second column.

Aspect 21 is the method of Aspect 19, wherein the separating step comprises: conveying the product mixture and an entrainer fluid to a first column; collecting a first distillate from the first column comprising a first component of the azeotrope or azeotrope-like composition; collecting a first bottoms product from the first column comprising a mixture of the entrainer and a second component of the azeotrope or azeotrope-like composition; conveying the first bottoms product to a second column to separate the entrainer and the second component of the azeotrope or azeotrope-like composition; and removing a composition consisting essentially of the second component of the azeotrope or azeotrope-like composition as a second distillate from the second column.

Aspect 22 is 1,1-difluoropropene (HFO-1252zc) produced by the method of Aspect 19.

What is claimed is:

1. An azeotrope or azeotrope-like composition consisting essentially of effective amounts of 1,1-difluoropropene (HFO-1252zc) and one of chlorotrifluoroethylene (CTFE), hexafluoropropylene (HFP), or cis-1,2-difluoroethylene (HFO-1132(Z)).

2. The azeotrope or azeotrope-like composition of claim 1, consisting essentially of 1,1-difluoropropene (HFO-1252zc) and chlorotrifluoroethylene (CTFE).

3. The azeotrope or azeotrope-like composition of claim 2, consisting essentially of from about 0.1 wt. % to about 99.9 wt. % of 1,1-difluoropropene (HFO-1252zc) and from about 0.1 wt. % to about 99.9 wt. % of chlorotrifluoroethylene (CTFE).

4. The azeotrope or azeotrope-like composition of claim 2, consisting essentially of from about 19.2 wt. % to about 99.9 wt. % of 1,1-difluoropropene (HFO-1252zc) and from about 0.1 wt. % to about 80.8 wt. % of chlorotrifluoroethylene (CTFE).

5. The azeotrope or azeotrope-like composition of claim 2, consisting essentially of about 57.5 wt. % of 1,1-difluoropropene (HFO-1252zc) and about 42.5 wt. % of chlorotrifluoroethylene (CTFE) at a pressure of about 358.8 psia.

6. The azeotrope or azeotrope-like composition of claim 2, wherein the azeotrope or azeotrope-like composition has a boiling point of from about −29.4° C. to about 78.8° C., at a pressure of about 15.6 psia to about 358.8 psia.

7. The azeotrope or azeotrope-like composition of claim 1, consisting essentially of 1,1-difluoropropene (HFO-1252zc) and hexafluoropropylene (HFP).

8. The azeotrope or azeotrope-like composition of claim 7, consisting essentially of from about 0.1 wt. % to about 99.9 wt. % of 1,1-difluoropropene (HFO-1252zc) and from about 0.1 wt. % to about 99.9 wt. % of hexafluoropropylene (HFP).

9. The azeotrope or azeotrope-like composition of claim 7, consisting essentially of from about 0.1 wt. % to about 57.5 wt. % of 1,1-difluoropropene (HFO-1252zc) and from about 42.5 wt. % to about 99.9 wt. % of hexafluoropropylene (HFP).

10. The azeotrope or azeotrope-like composition of claim 7, consisting essentially of from about 0.1 wt. % to about 41.7 wt. % of 1,1-difluoropropene (HFO-1252zc) and from about 58.3 wt. % to about 99.9 wt. % of hexafluoropropylene (HFP).

11. The azeotrope or azeotrope-like composition of claim 7, consisting essentially of about 23.9 wt. % of 1,1-difluoropropene (HFO-1252zc) and about 76.1 wt. % of hexafluoropropylene (HFP) at a pressure of about 404.1 psia.

12. The azeotrope or azeotrope-like composition of claim 1, consisting essentially of 1,1-difluoropropene (HFO-1252zc) and cis-1,2-difluoroethylene (HFO-1132(Z)).

13. The azeotrope or azeotrope-like composition of claim 12, consisting essentially of from about 0.1 wt. % to about 99.9 wt. % of 1,1-difluoropropene (HFO-1252zc) and from about 0.1 wt. % to about 99.9 wt. % of cis-1,2-difluoroethylene (HFO-1132(Z)).

14. The azeotrope or azeotrope-like composition of claim 12, consisting essentially of from about 34.4 wt. % to about 79.4 wt. % of 1,1-difluoropropene (HFO-1252zc) and from about 20.6 wt. % to about 65.6 wt. % of cis-1,2-difluoroethylene (HFO-1132(Z)).

15. The azeotrope or azeotrope-like composition of claim 12, consisting essentially of from about 44.9 wt. % to about 67.6 wt. % of 1,1-difluoropropene (HFO-1252zc) and from about 32.4 wt. % to about 55.1 wt. % of cis-1,2-difluoroethylene (HFO-1132(Z)).

16. The azeotrope or azeotrope-like composition of claim 12, consisting essentially of about 56.1 wt. % of 1,1-difluoropropene (HFO-1252zc) and about 43.9 wt. % of cis-1,2-difluoroethylene (HFO-1132(Z)) at a pressure of about 388.2 psia.

17. A method for producing 1,1-difluoropropene (HFO-1252zc) comprising: producing a product mixture comprising an azeotrope or azeotrope-like composition consisting essentially of effective amounts of 1,1-difluoropropene (HFO-1252zc) and any one of chlorotrifluoroethylene (CTFE), hexafluoropropylene (HFP), and/or cis-1,2-difluoroethylene (HFO-1132(Z)); and separating the 1,1-difluoropropene (HFO-1252zc) and the chlorotrifluoroethylene (CTFE), hexafluoropropylene (HFP), and/or cis-1,2-difluoroethylene (HFO-1132(Z)) to provide a product composition comprising the 1,1-difluoropropene (HFO-1252zc).

18. The method of claim 17, wherein the separating step comprises: conveying the product mixture to a first column having a first pressure; collecting a first bottoms product from the first column; conveying a first distillate from the first column to a second column having a second pressure to provide a second distillate and a second bottoms product, the second distillate comprising the azeotrope or azeotrope-like composition; and collecting a second bottoms product from the second column.

19. The method of claim 17, wherein the separating step comprises: conveying the product mixture and an entrainer fluid to a first column; collecting a first distillate from the first column comprising a first component of the azeotrope or azeotrope-like composition; collecting a first bottoms product from the first column comprising a mixture of the entrainer and a second component of the azeotrope or azeotrope-like composition; conveying the first bottoms product to a second column to separate the entrainer and the second component of the azeotrope or azeotrope-like composition; and removing a composition consisting essentially of the second component of the azeotrope or azeotrope-like composition as a second distillate from the second column.

20. 1,1-difluoropropene (HFO-1252zc) produced by the method of claim 17.
